(19) United States
(12) Patent Application Publication
(10) Pub. No.: US 2015/0017665 A1
(43) Pub. Date: Jan. 15, 2015

(54) Peptides and Methods for Detecting MPO-ANCA

(71) Applicant: The University of North Carolina at Chapel Hill, Chapel Hill, NC (US)

(72) Inventors: Ronald Falk, Chapel Hill, NC (US); Aleeza Roth, Chapel Hill, NC (US)

(73) Assignee: The University of North Carolina at Chapel Hill, Chapel Hill, NC (US)

(21) Appl. No.: 14/370,498

(22) PCT Filed: Jan. 3, 2013
(86) PCT No.: PCT/US13/20008

§ 371 Date: Jul. 3, 2014

Related U.S. Application Data
(60) Provisional application No. 61/582,729, filed on Jan. 3, 2012.

(51) Int. Cl. G01N 33/564; C12N 9/64; C12N 9/08

(52) U.S. Cl. CPC G01N 33/564; C12N 9/0065; C12N 9/6424; C12Y 111/02002; C12Y 304/21076; G01N 2800/347
USPC 435/7.92; 530/328; 530/327; 530/326; 530/329; 530/330; 436/501; 422/69; 435/287.2

(57) ABSTRACT

The present invention relates to disorder-specific epitopes recognized by myeloperoxidase-anti-neutrophil cytoplasmic autoantibodies and the use of polypeptides comprising the epitopes in methods of diagnosing and monitoring autoimmune disorders associated with the autoantibodies.

### STATEMENT OF PRIORITY

This application claims the benefit of U.S. Provisional Application Ser. No. 61/582,729, filed Jan. 3, 2012, the entire contents of which are incorporated by reference herein.

### STATEMENT OF FEDERAL SUPPORT

This invention was made with government support under grant number 2PO1DK58335-11 awarded by the National Institutes of Health. The government has certain rights in the invention.

### FIELD OF THE INVENTION

The present invention relates to disorder-specific epitopes recognized by myeloperoxidase-anti-neutrophil cytoplasmic autoantibodies and the use of polypeptides comprising the epitopes in methods of diagnosing and monitoring autoimmune disorders associated with the autoantibodies.

### BACKGROUND

Myeloperoxidase anti-neutrophil cytoplasmic autoantibodies (MPO-ANCA) and proteinase 3-specific ANCA (PR3-ANCA) are serologic markers used in routine clinical assays to diagnose small vessel necrotizing vasculitis (e.g., microscopic polyangiitis [MPA] and granulomatosis with polyangiitis [GPA] (Tervaert et al., Sarcoidosis Vasc. Diffuse Lung Dis. 13:241 (1996))). In vitro and in vivo studies provide compelling evidence that ANCA play a critical role in the pathogenesis of ANCA-associated vasculitis (AAV) (Falk et al., Proc. Natl. Acad. Sci. USA 87:4115 (1990); Xiao et al., J. Clin. Invest. 110:955 (2002)). However, three clinical observations plague the contention that ANCA are pathogenic. Firstly, conventional serologic assays fail to detect ANCA in some patients with classic clinical and pathologic features of AAV (Chen et al., Nature Rev. Nephrol. 5:313 (2009)). These patients are labeled as having ANCA-negative disease. Secondly, ANCA titers do not correlate well with disease activity, especially in MPO-ANCA disease (Hogan et al., J. Am. Soc. Nephrol. 7:23 (1996); Pagnoux et al., Arthritis Rheum. 58:2908 (2008)). Thirdly, naturally occurring anti-MPO and anti-PR3 antibodies exist in healthy individuals (Cui et al., Kidney Int. 78:590 (2010); Olson et al., J. Am. Soc. Nephrol. 22:1946 (2011)).

### SUMMARY OF EMBODIMENTS OF THE INVENTION

The present invention is based on the discovery of disorder-specific epitopes recognized by MPO-ANCA that explain the inconsistencies described above. The invention is further based on the discovery of an autoreactive antibody in ANCA-negative AAV whose detection is masked by a natural inhibitor of MPO. These discoveries provide improved methods of detecting, diagnosing, and monitoring autoimmune disorders associated with MPO-ANCA.

Thus, one aspect of the invention relates to an isolated polypeptide comprising a peptide as shown in Table 1 or an equivalent thereof that is specifically bound by a MPO-ANCA that specifically binds to the peptide of Table 1.

Another aspect of the invention relates to a method of detecting the presence of MPO-ANCA in a biological sample from a mammalian subject, the method comprising: contacting the sample with the polypeptide of the invention; and detecting binding of MPO-ANCA in the sample to the polypeptide, wherein binding of MPO-ANCA to the polypeptide indicates the presence of MPO-ANCA in the sample.

An additional aspect of the invention relates to a method of diagnosing a mammalian subject with an autoimmune disorder characterized by the presence of MPO-ANCA and/or determining if the subject is at risk (e.g., at increased risk) for the autoimmune disorder, the method comprising: contacting a biological sample from the subject with the polypeptide of the invention; and detecting binding of MPO-ANCA in the sample to the polypeptide, wherein binding of MPO-ANCA to the polypeptide diagnoses the subject with the autoimmune disorder and/or indicates that the subject is at risk (e.g., at increased risk) for the autoimmune disorder.

A further aspect of the invention relates to a method of monitoring disease progression in a mammalian subject with an autoimmune disorder characterized by the presence of MPO-ANCA, the method comprising: contacting a biological sample from the subject with the polypeptide of the invention; and detecting binding of MPO-ANCA in the sample to the polypeptide, wherein binding of MPO-ANCA to the polypeptide correlates with disease progression in the subject.

Another aspect of the invention relates to a method of determining whether a mammalian subject is in remission from an autoimmune disorder characterized by the presence of MPO-ANCA, the method comprising: contacting a biological sample from the subject with the polypeptide of the invention; and detecting binding of MPO-ANCA in the sample to the polypeptide, wherein binding of MPO-ANCA to the polypeptide provides information regarding whether the subject is in remission.

### BRIEF DESCRIPTION OF THE DRAWINGS

The accompanying drawings, which are incorporated in and constitute a part of the specification, illustrate embodiments of the invention and, together with the description, serve to explain principles of the invention.

FIGS. 1A-1D show a study of autoantibody epitope specificity within an MPO-ANCA positive cohort. FIG. 1A: Heat map demonstrating high level positive epitope binding (light gray), weakly positive epitope binding (low titer) detected only with very sensitive H₂¹⁸O labeling (dark gray) and negative detection (black) reactivity of n=97 immunoglobulin samples (y axis) with n=25 different peptide epitopes (x axis). The y axis includes samples from active MPO-ANCA patients (n=52), MPO-ANCA patients in remission (n=35), and healthy controls (n=10). Epitopes were designated exclusive to active disease if MPO-ANCA reactive with the epitopes came only from patients with active disease. Epitopes were designated persistent during remission if MPO-ANCA reactive with the epitopes occurred not only in active patients but also in patients in remission and rarely at low level in healthy controls. Epitopes were designated asymptomatic or “natural” if the epitopes were recognized by very low level autoantibodies detected with sensitive H₂¹⁸O labeling in healthy controls. FIG. 1B: Analysis of epitope specificity by MS was performed on Ig from a Chapel Hill (CH) cohort (n=97) and a European cohort from Groningen, The Netherlands (NL) (n=20). FIGS. 1B-1D show the distribution of autoantibody epitopes identified. CH-active disease (n=52), NL-active disease (n=20), clinical remission (n=35), healthy individuals (n=10). Unique epitopes strictly associated with ANCA disease were identified in all 72 active disease samples (FIGS. 1B and 1C). Extremely low level anti-MPO autoantibodies from healthy subjects were epitope mapped. Although detection required H₂¹⁸O labeling, none of these were reactive with disease specific epitopes (FIG. 1D).

FIG. 2 shows the Wilcoxon signed-rank test comparing the number of epitope specific autoantibodies with the age of the individual at the time of sample.

FIGS. 3A-3C show that MPO-ANCA reactive with epitope aa447-459 are exclusively associated with active disease. A single autoantibody (anti-MPO⁴⁴⁷⁴⁵⁹) associated with active ANCA disease was characterized as linear, by the fact that disruption of tertiary structure by pre-digestion did not affect antibody reactivity. MS data was validated by ELISA showing an association of this epitope with autoantibodies present in active disease. FIG. 3A underscores a correlation with disease activity utilizing longitudinal samples from individual patients over their disease course. Reactivity to both MPO epitope aa447-459 and aa516-524 was measured by OD values using a goat anti-human IgG specific secondary antibody. The correlation of anti-MPO⁴⁴⁷⁻⁴⁵⁹ autoantibodies with disease activity was recapitulated in a second independent cohort from Groningen, The Netherlands by ELISA (FIG. 3A). Asymptomatic anti-MPO⁵¹⁶⁻⁵²⁴ autoantibodies that are persistent during remission were also found to be linear and assayed by peptide ELISA (FIG. 3B). These antibodies were found in active disease and remission but were absent in healthy subjects in both cohorts (FIG. 3B). ELISAs involving the NL cohort were assayed at the University Medical Center Groningen using a different method. The epitopes associated exclusively with active ANCA disease (including aa447-459) exist in tandem with epitopes recognized by natural autoantibodies (FIG. 3C).

FIG. 4 shows conformationally dependent MPO epitopes, determined by loss of reactivity with pre-digested protein during epitope excision MS protocol, that were synthesized as peptides and tested for reactivity by ELISA.

FIGS. 5A-5C show epitope excision/MS detects a profile of autoantibodies in patients with an ANCA negative serology. IgG from seronegative CH patients (n=10) and NL patients (n=12) was incubated with leukocyte protein lysates as depicted in FIG. 5A. Reactive antigens were captured by autoantibodies. Sites of contact between the autoantigen and the IgG (epitope) were protected from digestion. Peptides remaining bound to IgG after digestion were eluted with 0.1% TFA and analyzed on a MALDI TOF/TOF MS/MS. Analysis of ANCA seronegative IgG yielded a single peak determined to be a linear MPO epitope aa447-459 (SEQ ID NO: 6) (FIG. 5B). Presence of anti-MPO autoantibodies was confirmed by ELISA probing with IgG for reactivity against native MPO, as compared to healthy subjects (FIGS. 5C and 5D). Specificity was confirmed by ELISA probing with IgG for reactivity against synthetic MPO peptide aa447-459 (FIGS. 5C and 5D). Longitudinal samples from CH cohort (n=4 individuals) and NL cohort (n=5 individuals) indicate a correlation between IgG reactivity, against MPO and synthetic MPO peptide aa447-459, with disease activity (FIGS. 5C and 5D).

FIGS. 6A-6H show that MPO epitope aa447-459 is masked by a proteolytic fragment of a common serum protein. FIG. 6A presents ELISA results comparing reactivity of sera to that of purified IgG from eight ANCA-negative vasculitis patients against native MPO as substrate. In FIG. 6B the substrate used was a synthetic peptide of epitope aa447-459. A putative serum factor was masking epitope aa447-459 from antibody binding, as shown by spiking sera into purified IgG (FIG. 6C). FIGS. 6D-6F show protein studies to identify the ‘masking’ factor in serum. Immobilized peptide aa447-459 was used to fish out putative proteins that bound to this specific sequence. A SDS-PAGE, Coomassie stained gel (FIG. 6D) of eluted serum proteins revealed a prominent band at ˜50 kD. The protein was excised, sequenced and identified as a fragment of ceruloplasmin (CP). Identity was confirmed by western blot (FIG. 6E) probed with an anti-CP antibody. Purified CP was purchased and digested with plasmin in vitro to produce a 50 kD fragment (FIG. 6F). ELISA results (FIG. 6G) indicated that full-length CP (151 kD) did not mask the epitope, while CP cleaved by plasmin was affective in blocking reactivity by 30-50%. Reactivity appears unaffected by addition of undigested CP to MPO-ANCA IgG (polyclonal) from four patients (FIG. 6H) indicating the specificity of the CP fragment masking effect on aa447-459.

FIG. 7 shows in vitro and in vivo pathogenic potential of anti-MPO⁴⁴⁷⁻⁴⁵⁹. In vitro pathogenic potential can be measured by the ability of ANCA to induce neutrophils to release reactive oxygen species. As displayed in the bar graph, affinity purified anti-MPO⁴⁴⁷⁻⁴⁵⁹ autoantibodies are capable of activating neutrophils while nonpathogenic (516-524) and natural (579-590, 237-248, and 530-536) anti-MPO autoantibodies were not. Neutrophils isolated from healthy subjects (n=4) were exposed to purified autoantibodies from unique individuals (n=4). Error bars represent 2 standard deviations above and below the mean of replicate experiments (n=4), further statistics were not done due to the limited sample size.

FIGS. 8A-8H show that DR2 tg mice immunized with MPO epitope aa447-459 develop glomerulonephritis. FIG. 8A shows indirect immunofluorescence of thioglycolate induced peritoneal neutrophils using pooled serum IgG of DR2 tg mice immunized with the MPO B epitope (IgG concentration for staining: 1 mg/ml). FIG. 8B shows albuminuria measured by ELISA on urine collected 24 hours before the end of the experiment. FIG. 8C shows haematuria measured by urine test strips. FIGS. 8D, 8E, and 8F show abnormal glomeruli assessed on formalin fixed PAS stained kidneys. Abnormalities assessed were thickening of the GBM (FIG. 8E) and evidence of mesangial hypercellularity (three or more attached nuclei) (FIG. 8F). Necrosis was rare. FIG. 8G shows glomerular hypercellularity assessed by enumerating the number of cells per glomerulus. FIG. 8H shows glomerular neutrophil recruitment assessed by immunohistochemistry by anti-Gr-1 antibody on PLP-fixed frozen kidneys.

FIGS. 9A-9H show that passive transfer of IgG from MPO peptide aa442-460 immunized mice is nephritogenic. Independently, a T cell MPO epitope had been identified, and DR2 transgenic mice were injected with the overlapping MPO peptide aa442-460 (LYQEARKIVGAMVQIITYR SEQ ID NO: 35)) that includes the human MPO epitope aa447-459 (RKIVGAMVQIITY (SEQ ID NO: 6)). Albuminuria and haematuria (FIGS. 9A and 9B) were measured on day 1 and 6. Blood urea nitrogen (BUN) was analyzed by a standard laboratory colorimetric assay (FIG. 9C). Abnormal glomeruli (day 6, FIGS. 9D, 9G and 9H) were assessed based on capillary wall thickening and mesangial hypercellularity on formalin fixed PAS stained kidney sections (scale bar=45 μm). Glomerular hypercellularity was assessed by enumerating the number of cells per glomerulus at day 6 (FIG. 9E). Neutrophil recruitment (FIG. 9F) was assessed based on immunohistochemistry by anti-Gr-1 antibodies on PLP-fixed frozen kidneys.

### DETAILED DESCRIPTION OF EMBODIMENTS OF THE INVENTION

The present invention now will be described hereinafter with reference to the accompanying drawings and examples, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

### DEFINITIONS

As used herein, “a,” “an,” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of dose (e.g., an amount of a peptide) and the like, is meant to encompass variations of ±20%, ±10%, ±5%, ±1%, ±0.5%, or even ±0.1% of the specified amount.

The terms “comprise,” “comprises,” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” when used in a claim or the description of this invention is not intended to be interpreted to be equivalent to “comprising.”

As used herein, the terms “increase,” “increases,” “increased,” “increasing,” and similar terms indicate an elevation of at least about 25%, 50%, 75%, 100%, 150%, 200%, 300%, 400%, 500% or more.

As used herein, the terms “reduce,” “reduces,” “reduced,” “reduction,” and similar terms mean a decrease of at least about 5%, 10%, 15%, 20%, 25%, 35%, 50%, 75%, 80%, 85%, 90%, 95%, 97% or more. In particular embodiments, the reduction results in no or essentially no (i.e., an insignificant amount, e.g., less than about 10% or even 5%) detectable activity or amount.

As used herein, the term “nucleic acid” encompasses both RNA and DNA, including cDNA, genomic DNA, synthetic (e.g., chemically synthesized) DNA and chimeras of RNA and DNA. The nucleic acid may be double-stranded or single-stranded. The nucleic acid may be synthesized using nucleotide analogs or derivatives (e.g., inosine or phosphorothioate nucleotides). Such nucleotides can be used, for example, to prepare nucleic acids that have altered base-pairing abilities or increased resistance to nucleases.

As used herein, the term “polypeptide” encompasses both peptides and proteins (including fusion proteins), unless indicated otherwise.

A “fusion protein” is a polypeptide produced when two heterologous nucleotide sequences or fragments thereof coding for two (or more) different polypeptides not found fused together in nature are fused together in the correct translational reading frame.

Two amino acid sequences are said to be “substantially identical” or “substantially similar” to each other when they share at least about 50%, 60%, 70%, 75%, 80%, 85%, 90%, 95%, 97%, 98%, 99% or even more sequence identity or similarity, respectively.

As used herein “sequence identity” refers to the extent to which two optimally aligned polynucleotide or polypeptide sequences are invariant throughout a window of alignment of components, e.g., nucleotides or amino acids.

As used herein “sequence similarity” is similar to sequence identity (as described herein), but permits the substitution of conserved amino acids (e.g., amino acids whose side chains have similar structural and/or biochemical properties), which are well-known in the art.

As is known in the art, a number of different programs can be used to identify whether a nucleic acid has sequence identity or an amino acid sequence has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp, CABIOS 5:151-153 (1989).

Another example of a useful algorithm is the BLAST algorithm, described in Altschul et al., J. Mol. Biol. 215:403-410, (1990) and Karlin et al., Proc. Natl. Acad. Sci. USA 90:5873-5787 (1993). A particularly useful BLAST program is the WU-BLAST-2 program which was obtained from Altschul et al., Methods in Enzymology 266:460-480 (1996); http://blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al. Nucleic Acids Res. 25:3389-3402 (1997).

The CLUSTAL program can also be used to determine sequence similarity. This algorithm is described by Higgins et al. Gene 73:237 (1988); Higgins et al. CABIOS 5:151-153 (1989); Corpet et al. Nucleic Acids Res. 16:10881-90 (1988); Huang et al. CABIOS 8:155-65 (1992); and Pearson et al. Meth. Mol. Biol. 24:307-331 (1994).

The alignment may include the introduction of gaps in the sequences to be aligned. In addition, for sequences which contain either more or fewer nucleotides than the nucleic acids disclosed herein, it is understood that in one embodiment, the percentage of sequence identity will be determined based on the number of identical nucleotides acids in relation to the total number of nucleotide bases. Thus, for example, sequence identity of sequences shorter than a sequence specifically disclosed herein, will be determined using the number of nucleotide bases in the shorter sequence, in one embodiment. In percent identity calculations relative weight is not assigned to various manifestations of sequence variation, such as, insertions, deletions, substitutions, etc.

The polypeptides of the invention are optionally “isolated.” An “isolated” polypeptide is a polypeptide that, by the hand of man, exists apart from its native environment and is therefore not a product of nature. An isolated polypeptide may exist in a purified form or may exist in a non-native environment such as, for example, a recombinant host cell. Recombinant polypeptides produced using recombinant nucleic acid techniques can be considered to be “isolated.”

In representative embodiments, an “isolated” polypeptide means a polypeptide that is separated or substantially free from at least some of the other components of the naturally occurring organism or virus, for example, the cell or viral structural components or other polypeptides or nucleic acids commonly found associated with the polypeptide. In particular embodiments, the “isolated” polypeptide is at least about 1%, 5%, 10%, 25%, 50%, 60%, 70%, 75%, 80%, 85%, 90%, 95%, 97%, 98%, 99% or more pure (w/w). In other embodiments, an “isolated” polypeptide indicates that at least about a 5-fold, 10-fold, 25-fold, 100-fold, 1000-fold, 10,000-fold, or more enrichment of the protein (w/w) is achieved as compared with the starting material. In representative embodiments, the isolated polypeptide is a recombinant polypeptide produced using recombinant nucleic acid techniques.

The term “fragment,” as applied to a polypeptide of the invention, will be understood to mean an amino acid of reduced length relative to a reference polypeptide or the full-length polypeptide (e.g., MPO epitope) and comprising, consisting essentially of, and/or consisting of a sequence of contiguous amino acids from the reference or full-length polypeptide. Such a fragment according to the invention may be, where appropriate, included as part of a fusion protein of which it is a constituent. In some embodiments, such fragments can comprise, consist essentially of, and/or consist of polypeptides having a length of at least about 4, 5, 6, 7, 8, 9, or 10 amino acids (optionally, contiguous amino acids) from the reference or full-length polypeptide, as long as the fragment is shorter than the reference or full-length polypeptide. In embodiments of the invention, the fragment is less than about 30, 25, 20, 18, 15, 12, 10, 9, 8 7, 6, or 5 amino acids (optionally, contiguous amino acids) from the reference or full-length polypeptide. In representative embodiments, the fragment is bound (e.g., specifically binds) by the MPO-ANCA that binds to the full-length or reference polypeptide (e.g., MPO epitope).

“Effective amount” as used herein refers to an amount of a vector, nucleic acid, polypeptide, composition and/or formulation of the invention that is sufficient to produce a desired effect, which can be a therapeutic and/or beneficial effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation.

By the term “treat,” “treating,” or “treatment of” (and grammatical variations thereof) it is meant that the severity of the subject's condition is reduced, at least partially improved or ameliorated and/or that some alleviation, mitigation or decrease in at least one clinical symptom is achieved and/or there is a delay in the progression of the disease or disorder.

A “treatment effective” amount as used herein is an amount that is sufficient to treat (as defined herein) the subject. Those skilled in the art will appreciate that the therapeutic effects need not be complete or curative, as long as some benefit is provided to the subject.

The term “prevent,” “preventing,” or “prevention of” (and grammatical variations thereof) refer to prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of the present invention.

A “prevention effective” amount as used herein is an amount that is sufficient to prevent (as defined herein) the disease, disorder and/or clinical symptom in the subject. Those skilled in the art will appreciate that the level of prevention need not be complete, as long as some benefit is provided to the subject.

A “diagnostic method,” as used herein, refers to a screening procedure that is carried out to identify those subjects that are affected or likely to be affected with a particular disorder. A “diagnostic method” need not be definitive or conclusive in identifying a subject and may be carried out in conjunction with, preceded and/or followed up by additional diagnostic measures.

A “prognostic method” refers to a method used to predict, at least in part, the course and/or severity of a disease. For example, a prognostic method may be carried out to both identify an affected individual, to evaluate the severity of the disease, and/or to predict the future course of the disease. Such methods may be useful in evaluating the necessity for therapeutic treatment, what type of treatment to implement, and the like. In addition, a prognostic method may be carried out on a subject previously diagnosed with a particular disorder when it is desired to gain greater insight into how the disease will progress for that particular subject and/or the likelihood that a particular patient will respond favorably to a particular drug treatment, or when it is desired to classify or separate patients into distinct and different sub-populations for the purpose of treatment and/or conducting a clinical trial. A “prognostic method” need not be definitive or conclusive and may be carried out in conjunction with, preceded and/or or followed up by additional prognostic measures.

A “subject” of the invention includes any animal susceptible to an autoimmune disorder characterized by the presence of MPO-ANCA and includes mammalian and avian subjects. Such a subject is generally a mammalian subject (e.g., a laboratory animal such as a rat, mouse, guinea pig, rabbit, primate, etc.), a farm or commercial animal (e.g., a cow, horse, goat, donkey, sheep, etc.), or a domestic animal (e.g., cat, dog, ferret, etc.). In particular embodiments, the subject is a primate subject, a non-human primate subject (e.g., a chimpanzee, baboon, monkey, gorilla, etc.) or a human. Inc certain embodiments, subjects of the invention can be a subject known to have or believed to have MPO-ANCA. Subjects of the invention can be a subject known or believed to be at risk of developing MPO-ANCA or an autoimmune disorder characterized by the presence of MPO-ANCA. Alternatively, a subject according to the invention can also include a subject not previously known or suspected to have MPO-ANCA or an autoimmune disorder characterized by the presence of MPO-ANCA. Subjects include males and/or females of any age, including neonates, juvenile, mature and geriatric subjects.

In embodiments of the invention the subject has or is suspected of having MPO-ANCA glomerulonephritis.

In embodiments of the invention, the subject has or is suspected of having ANCA-negative glomerulonephritis, e.g., no PR3-ANCA or MPO-ANCA have been detected by at least one other methodology.

A “subject in need” of the methods of the invention can be a subject known, suspected of having, or having an increased risk of developing MPO-ANCA or an autoimmune disorder characterized by the presence of MPO-ANCA.

“MPO-ANCA” refers to myeloperoxidase anti-neutrophil cytoplasmic autoantibody. As is well-known in the art, naturally occurring MPO-ANCA are generally polyclonal, i.e., directed against multiple epitopes on MPO. Unless the context indicates otherwise, as used herein, the term can refer to both polyclonal MPO-ANCA (e.g., containing antibodies of one or more epitope specificities) and/or can refer to MPO-ANCA directed against a single epitope, which may also be monoclonal or polyclonal. MPO-ANCA can be from any class of immunoglobulin, including IgG, IgA and/or IgM.

An autoimmune disorder characterized by the presence of MPO-ANCA can be any disorder or disease in which MPO-ANCA are present in the subject (e.g., elevated levels of MPO-ANCA as compared with a suitable control), for example, pathogenic or disease-associated MPO-ANCA. Such disorders are generally classified as auto-immune disorders and include without limitation, systemic vasculitides (sometimes called small vessel vasculitis), which feature autoimmune attacks against small and medium-sized blood vessels and which further includes, without limitation, Churg-Strauss Syndrome, microscopic polyangiitis (MPA), granulomatosis with polyangiitis (GPA) (also known as Wegener's granulomatosis) and/or pauci-immune necrotizing and crescentic glomerulonephritis.

As a first aspect, the invention provides a polypeptide (e.g., isolated polypeptide) comprising, consisting essentially of, or consisting of a peptide as shown in Table 1 or an equivalent thereof that is bound (e.g., specifically bound) by a MPO-ANCA that binds (e.g., specifically binds) to the peptide of Table 1. In representative embodiments, the peptide from Table 1 comprises a linear epitope.

TABLE 1
Common MPO-ANCA Epitopes of Active ANCA Patients

| | | Found in samples: | | |
|---|---|---|---|---|
| | | Healthy control, | | |
| Epitope AA | | Active disease, or | | |
| Sequence | Epitopes found | Remission | Epitope | SEQ |
| Number | in Humans | (HC, A, R) | Structure | ID NO |
| 490-499 | IANVFTNAFR | A | Conformational | 1 |
| 537-548 | VVLEGGIDPILR | A | Conformational | 2 |
| 328-351 | NQINALTSFVDASMV | A | Conformational | 3 |
| 220-228 | NGFPVALAR | A | Conformational | 4 |
| 198-219 | WLPAEYEDGFSLPYG | A | Conformational | 5 |
| 447-459 | RKIVGAMVQIITY | A | Linear | 6 |
| 369-374 | FQDNGR | A | Conformational | 7 |
| 184-193 | RSPTLGASNR | A | Conformational | 8 |
| 605-622 | FCGLPQPETVGQLGT | A | Conformational | 9 |
| 442-447 | LYQEAR | A | Conformational | 10 |
| 715-725 | NNIFMSNTYPR | A | Conformational | 11 |
| 657-664 | VGPLLACI | A | Conformational | 12 |
| 560-571 | QNQIAVDEIR | A, R | Conformational | 13 |
| 692-701 | QALAQISLPR | A ,R | Conformational | 14 |
| 474-480 | KYLPTYR | A ,R | Conformational | 15 |
| 437-441 | WDGER | A, R | Conformational | 16 |
| 396-405 | IPCFLAGDTR | R | Conformational | 17 |
| 516-524 | YQPMEPNPR | A, R, HC | Linear | 18 |
| 579-590 | IGLDLPALNMQR | A, R, HC | Linear | 19 |
| 530-536 | VFFASWR | A, R, HC | Linear | 20 |
| 237-248 | FPTDQLTPDQER | A, R, HC | Linear | 21 |
| 460-473 | RDYLPLVLGPTAMR | A, R, HC | Conformational | 22 |
| 593-603 | DHGLPGYNAWR | A, R, HC | Conformational | 23 |
| 516-524 | YQPMEPNPR | A, R, HC | Linear | 24 |
| 572-578 | LFEQVMR | A, R, HC | Conformational | 25 |
| 678-691 | FWWENEGVFSMQQR | A, R, HC | Conformational | 26 |

In representative embodiments, the polypeptide comprises a MPO peptide consisting or consisting essentially of a peptide as shown in Table 1 or an equivalent thereof that is bound by a MPO-ANCA that binds to the peptide of Table 1. In certain embodiments, the polypeptide is not full length MPO or wild-type MPO.

In representative embodiments, the polypeptide is a fusion protein, e.g., the polypeptide comprises a polypeptide that facilitates purification or detection. For example, the fusion protein can comprise hemagglutinin antigen, polyHis, biotin, Protein A, streptavidin, maltose binding protein, c-myc, FLAG, or an enzyme such as glutathione-S-transferase, alkaline phosphatase, horseradish peroxidase, β-glucuronidase, β-galactosidase and/or luciferase.

The polypeptide can optionally comprise a detectable moiety. In representative embodiments, the detectable moiety can be an exogenous epitope or chemical label that is covalently attached to the polypeptide. The detectable moiety can be any exogenous label that can be detected using any method known in the art. According to this embodiment, the detectable moiety can be an epitope, an enzyme, a ligand, a receptor, an antibody or antibody fragment and the like. In representative embodiments, the detectable moiety is a hemagglutinin antigen, polyHis, biotin, Protein A, streptavidin, maltose binding protein, c-myc, FLAG, or an enzyme such as glutathione-S-transferase, alkaline phosphatase, horseradish peroxidase, β-glucuronidase, β-galactosidase or luciferase. Further, the detectable moiety can be, without limitation, a fluorescent moiety (e.g., Green Fluorescent Protein or a nanocrystal (e.g., a quantum dot such as a Qdot® Nanocrystal from Invitrogen), a radioactive moiety and/or an electron-dense moiety such as a ferritin or gold particle(s).

The detectable moiety can be detected either directly or indirectly using any suitable method. For example, for direct detection, the detectable moiety can comprise a radioisotope (e.g., ³⁵S) and the presence of the radioisotope can be detected by autoradiography. As another example, the detectable moiety can comprise a fluorescent moiety and can be detected by fluorescence as is known in the art. Alternatively, the detectable moiety can be indirectly detected, i.e., the detectable moiety requires additional reagents to render it detectable. Illustrative methods of indirect labeling include those utilizing chemiluminescence agents, chromogenic agents, enzymes that produce visible reaction products, and ligands (e.g., haptens, antibodies or antigens) that may be detected by binding to labeled specific binding partners (e.g., hapten binding to a labeled antibody or a first antibody binding to a second antibody).

In particular embodiments, the detectable moiety is an antibody or antibody fragment. A variety of protocols for detecting the presence of and/or measuring the amount of antibodies or other polypeptides are known in the art. Examples of such protocols include, but are not limited to, enzyme-linked immunosorbent assays (ELISA), radioimmunoassay (RIA), radioreceptor assay (RRA), competitive binding assays and immunofluorescence. These and other assays are described, among other places, in Hampton et al. (Serological Methods, a Laboratory Manual, APS Press, St Paul, Minn. (1990)) and Maddox et al. (J. Exp. Med. 158:1211-1216 (1993)).

As is known in the art, the polypeptide can be isolated or purified from a biological source (e.g., blood), can be produced using recombinant nucleic acid techniques and/or can be wholly or partially synthetic. Chemically synthesized polypeptides can optionally comprise modified amino acids and/or linkages, e.g., to reduce susceptibility to proteases.

Equivalents of the peptides of Table 1 can be readily identified by those skilled in the art and include peptides that have substantially identical or similar amino acid sequences (as described herein) to the MPO peptides of Table 1 and fragments thereof. Further, the term equivalent includes homologous peptides from a human MPO or an ortholog from any other species including mammalian species (e.g., mice) and also includes naturally occurring allelic variations, isoforms, splice variants and the like. For example, it is known in the art that there are several variants of human MPO. The MPO peptides in Table 1 and the examples are numbered with respect to the human MPO sequence at GI 34719 (NCBI Accession No. CAA33438; mass=83801 daltons) and listed below as SEQ ID NO: 37. In embodiments of the invention, the equivalent is a human MPO equivalent (including fragments).

(SEQ ID NO: 37)

| | |
|---|---|
| 1 | mgvpffsslr cmvdlgpcwa ggltaemkll lalagvlail |
| | atpqpsegaa |
| 51 | pavlgevdts lvlssmeeak qlvdkayker resikqrlrs |
| | gsaspmells |
| 101 | yfkqpvaatr tavraadylh valdllerkl rslwrrpfnv |
| | tdvltpaqln |
| 151 | vlskssgcay qdvgvtcpeq dkyrtitgmc nnrrsptlga |
| | snrafvrwlp |
| 201 | aeyedgfslp ygwtpgvkrn gfpvalarav sneivrfptd |
| | qltpdqersl |
| 251 | mfmqwgqlld hdldftpepa arasfvtgvn cetscvqqpp |
| | cfplkippnd |
| 301 | priknqadci pffrscpacp gsnitirnqi naltsfvdas |
| | mvygseepla |
| 351 | rnlrnmsnql gllavnqrfq dngrallpfd nlhddpcllt |
| | nrsaripcfl |
| 401 | agdtrssemp eltsmhtlll rehnrlatel kslnprwdge |
| | rlyqearkiv |
| 451 | gamvqiityr dylplvlgpt amrkylptyr syndsvdpri |
| | anvftnafry |
| 501 | ghtliqpfmf rldnryqpme pnprvplsrv ffaswrvvle |
| | ggidpilrgl |
| 551 | matpaklnrq nqiavdeire rlfeqvmrig ldlpalnmqr |
| | srdhglpgyn |
| 601 | awrrfcglpq petvgqlgtv lrnlklarkl meqygtpnni |
| | diwmggvsep |
| 651 | lkrkgrvgpl laciigtqfr klrdgdrfww enegvfsmqq |
| | rqalaqislp |
| 701 | riicdntgit tvsknnifms nsyprdfvnc stlpalnlas |
| | wreas |

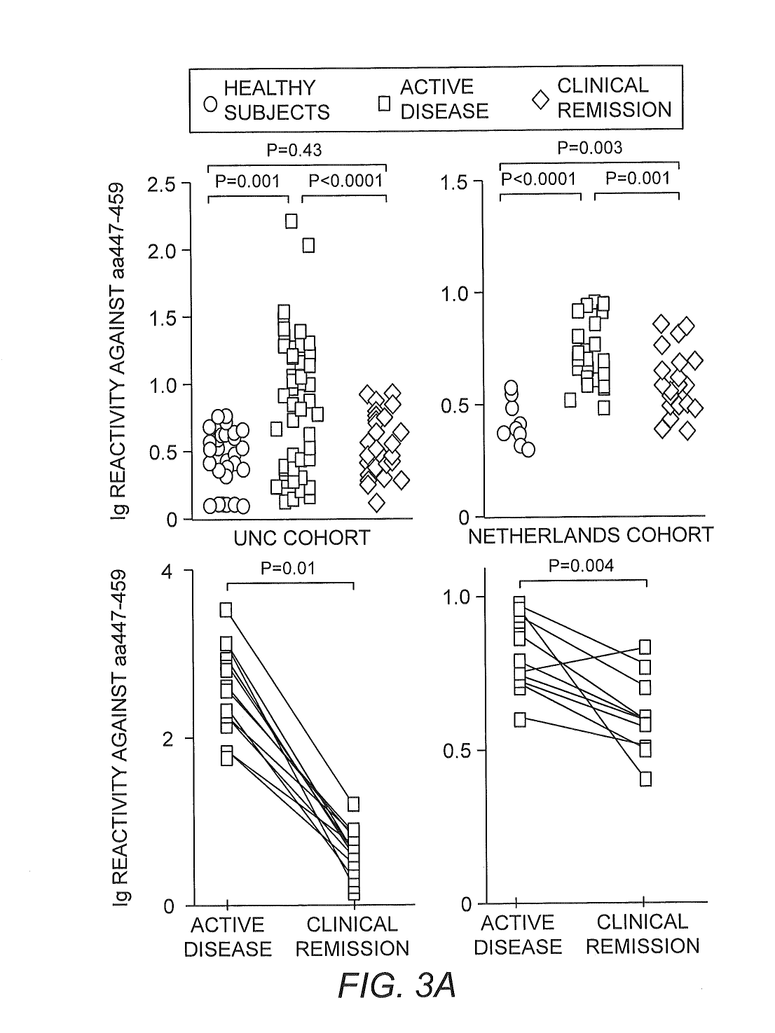

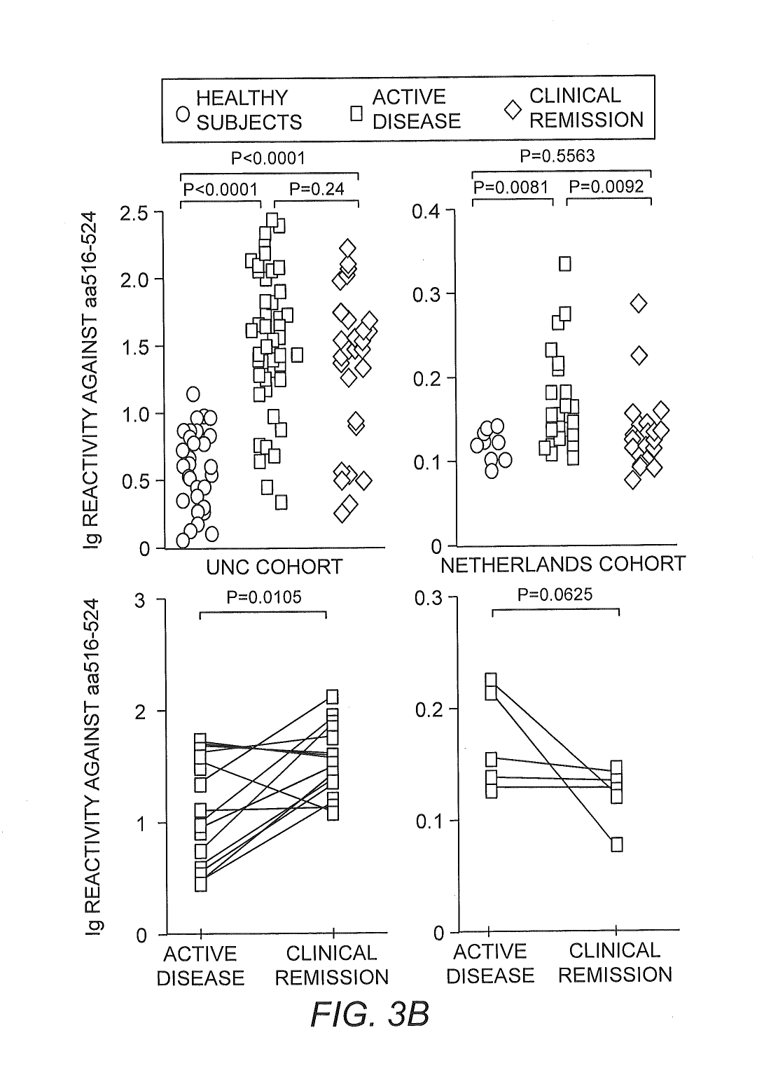

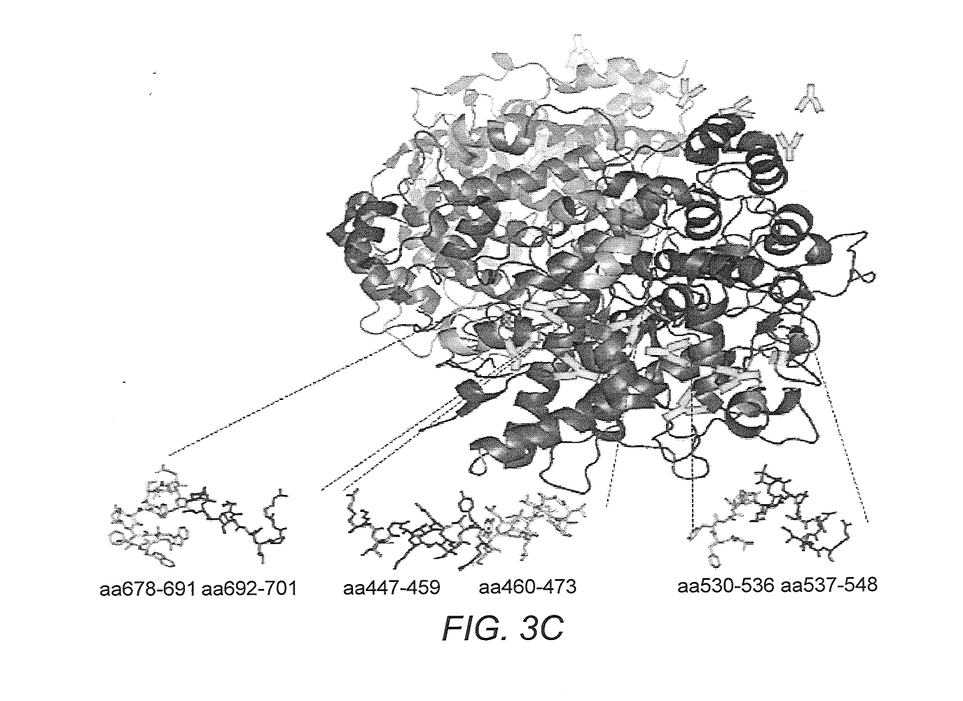

Equivalents of the peptides of Table I encompass those that have substantial amino acid sequence identity or similarity, for example, at least about 60%, 70%, 75%, 80%, 85%, 90%, 95%, 97%, 98%, 99% or more amino acid sequence identity or similarity with the amino acid sequences specifically disclosed herein (e.g., in Table 1) or a fragment thereof. In representative embodiments, the equivalent comprises the amino acid residues highlighted in FIG. 3 (for example, RKXVGA (SEQ ID NO: 27) or RKIVGA (SEQ ID NO: 28) from peptide 447-489 or VVLEG (SEQ ID NO: 29) from peptide 537-548), e.g., any sequence variability occurs outside these regions or there are only conservative substitutions in these regions.

It will further be understood that the MPO peptides specifically disclosed herein will typically tolerate substitutions in the amino acid sequence and substantially retain biological activity. To routinely identify peptides of the invention that substantially retain the ability to bind to ANCA other than those specifically disclosed herein, amino acid substitutions may be based on any characteristic known in the art, including the relative similarity or differences of the amino acid side-chain substituents, for example, their hydrophobicity, hydrophilicity, charge, size, and the like. In particular embodiments, conservative substitutions (i.e., substitution with an amino acid residue having similar properties) are made in the amino acid sequence encoding the peptide of Table 1 or fragment thereof.

In making amino acid substitutions, the hydropathic index of amino acids can be considered. The importance of the hydropathic amino acid index in conferring interactive biologic function on a protein is generally understood in the art (see, Kyte and Doolittle J. Mol. Biol. 157:105 (1982)). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of its hydrophobicity and charge characteristics (Kyte and Doolittle, Id.), and these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is also understood in the art that the substitution of amino acids can be made on the basis of hydrophilicity. U.S. Pat. No. 4,554,101 states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (±3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In representative embodiments, an equivalent of a peptide as described herein comprises 1, 2 or less, 3 or less, 4 or less, 5 or less, or 6 or less amino acid substitutions, insertions (including N-terminal and/or C-terminal extensions) and/or deletions (including truncations) as compared with a peptide of Table 1. Substitutions are optionally conservative substitutions.

In embodiments of the invention, the polypeptide comprising, consisting essentially of, or consisting of a peptide of Table 1 has a length of no more than 100 amino acids, e.g., no more than 90, 80, 70, 60, 50, 40, 30, 25, 20, 15, or 10 amino acids.

In embodiments of the invention, the polypeptide comprising, consisting essentially of, or consisting of a peptide of Table 1 includes additional MPO sequence at the N-terminal and/or C-terminal ends, for example, 1, 2 or less, 3 or less, 4 or less, 5 or less, 6 or less, 7 or less, 8 or less, 9 or less, 10 or less, 12 or less, 15 or less, 18 or less, 20 or less, 25 or less, 30 or less, 35 or less, 40 or less, 50 or less, 60 or less, or 100 or less additional contiguous MPO amino acids extending in the N-terminal and/or C-terminal direction with respect to the peptide of Table 1 as it exists in the native MPO protein. In certain embodiments, the polypeptide comprises both additional MPO sequence and non-MPO sequence at the N-terminal and/or C-terminal ends.

In embodiments of the invention, the polypeptide is not a MPO peptide as described in Tomizawa et al., J. Clin. Immunol. 18:142 (1998), Olson et al., J. Am. Soc. Nephrol. 22:1946 (2011), Xiao et al., J. Clin. Invest. 110:955 (2002), Chang et al., Clin. Exp. Immunol. 102:112 (1995), Bruner et al., Clin. Exp. Immunol. 164:330 (2011), and/or Specks, APMIS 117 (Suppl. 127):63 (2009).

In embodiments of the invention, an equivalent of a peptide of Table 1 is not RLDNRYQPMEPN (SEQ ID NO: 30) or, optionally, a fragment thereof (e.g., YQPMEPN (SEQ ID NO: 31) or any smaller fragment thereof). In embodiments of the invention, the MPO peptide does not comprise, consist essentially of, or consist of amino acids 165-272, 165-214, 215-272, 279-745, 269-731, 279-511, 512-745, 279-409, 341-474, 410-537, 512-598, 541-745 and/or 597-745 of human MPO or the homologous epitopes in other human MPO proteins or orthologs from other species.

In embodiments of the invention, the polypeptide does not encompass any portion of a non-disease associated MPO epitope that is recognized by ANCA. In embodiments of the invention, the MPO peptide does not encompass a complete non-disease associated MPO epitope that is recognized by ANCA. Non-disease associated epitopes include, e.g., amino acids 579-590, 237-248, 460-473, 530-536, 593-603, 572-578, 516-524, and/or 678-691 of human MPO (NCBI Accession No, CAA33438). The amino acid sequences of these epitopes are shown in Table 1.

The present invention also contemplates the use of the polypeptides of the invention, e.g., to detect and/or purify MPO-ANCA.

As one aspect, the invention provides a method of detecting the presence of MPO-ANCA in a biological sample from a subject, the method comprising: contacting the sample with a polypeptide of the invention; and detecting binding (e.g., specific binding) of MPO-ANCA in the sample to the polypeptide, wherein binding of MPO-ANCA to the polypeptide indicates the presence of MPO-ANCA that binds to the peptide in the sample. In representative embodiments, the method comprises detecting the presence or absence of binding (e.g., specific binding) of MPO-ANCA in the sample to the polypeptide.

The invention also provides a method of diagnosing a subject with an autoimmune disorder characterized by the presence of MPO-ANCA and/or determining if the subject is at risk for the autoimmune disorder, the method comprising: contacting a biological sample from the subject with a polypeptide of the invention; and detecting binding (e.g., specific binding) of MPO-ANCA in the sample to the polypeptide, wherein binding of MPO-ANCA to the polypeptide diagnoses the subject with the autoimmune disorder and/or indicates that the subject is at risk (e.g., increased risk) for the autoimmune disorder. Increased risk can be determined with respect to the level of risk in a suitable control, e.g., the same subject at an earlier time, a different subject that does not have a MPO-ANCA related disorder, or a population of subjects that do not have a MPO-ANCA related disorder. In representative embodiments, the method comprises detecting the presence or absence of binding (e.g., specific binding) of MPO-ANCA in the sample to the polypeptide.

The invention further provides a method of monitoring disease progression in a subject with an autoimmune disorder characterized by the presence of MPO-ANCA, the method comprising: contacting a biological sample from the subject with a polypeptide of the invention; and detecting binding (e.g., specific binding) of MPO-ANCA in the sample to the polypeptide, wherein binding of MPO-ANCA to the polypeptide correlates with disease progression in the subject. In representative embodiments, the method comprises detecting the presence or absence of binding (e.g., specific binding) of MPO-ANCA in the sample to the polypeptide. According to this aspect, the invention can be used to monitor improvement and/or increasing severity of the subject's condition and/or can be used to detect and/or monitor remission, active disease and/or relapse in the subject. Generally, an increased level of MPO-ANCA compared to a suitable control correlates with active disease, relapse, and/or increased severity of condition while a lower level as compared with the level of MPO-ANCA in active disease correlates with remission and/or decreased severity of condition.

Without being limited by any theory of the invention, although total MPO-ANCA titers as currently assessed may not correlate well with disease severity, in representative embodiments, MPO-ANCA directed against the peptides and equivalents thereof of the invention may correlate better with clinical disease.

The invention further provides a method of determining whether a subject is in remission from an autoimmune disorder characterized by the presence of MPO-ANCA, the method comprising: contacting a biological sample from the subject with a polypeptide of the invention; and detecting binding (e.g., specific binding) of MPO-ANCA in the sample to the polypeptide, wherein binding of MPO-ANCA to the polypeptide provides information regarding whether the subject is in remission. In representative embodiments, the method comprises detecting the presence or absence of binding (e.g., specific binding) of MPO-ANCA in the sample to the polypeptide. Generally, an absence or low level of MPO-ANCA that binds to the polypeptide as compared with the level of MPO-ANCA in active disease indicates that the subject is in remission.

The invention also provides a method of determining whether a subject has experienced a relapse in an autoimmune disorder characterized by the presence of MPO-ANCA, the method comprising: contacting a biological sample from the subject with a polypeptide of the invention; and detecting binding (e.g., specific binding) of MPO-ANCA in the sample to the polypeptide, wherein binding of MPO-ANCA to the polypeptide provides information regarding whether the subject has relapsed. In representative embodiments, the method comprises detecting the presence or absence of binding (e.g., specific binding) of MPO-ANCA in the sample to the polypeptide. Generally, an increase in the level of MPO-ANCA as compared with the level of MPO-ANCA in the subject during remission indicates relapse of the disease.

In each of the methods of the invention, the method can comprise further steps such as treatment and/or monitoring based on the outcome of the assay. If the presence of MPO-ANCA is detected in a biological sample from a subject, in one embodiment a further step can be initiation of treatment. In another embodiment, a further step can be continued monitoring of the subject for an increase in MPO-ANCA levels to a level at which treatment is deemed necessary or for a decrease in MPO-ANCA levels. If a subject is determined to be in remission, a further step can include terminating treatment, decreasing the intensity of treatment, or switching to a different treatment. If a subject is determined to have relapsed, a further step can include initiating treatment, increasing the intensity of treatment, or switching to a different treatment.

Treatments for MPO-ANCA-related disorders are known in the art and can include, without limitation, immunosuppressive drugs such as corticosteroids, cyclophosphamide, co-trimoxazole, mycophenolate mofetil, 15-deoxyspergualin, anti-thymocyte globulin, rituximab, and infliximab. After remission treatment can be changed, for example, to azathioprine, methotrexate, and/or tapered doses of corticosteroids. Plasmapheresis to remove MPO-ANCA may also be included in treatment. Treatment for symptoms related to the disorders may also be included.

In the methods of the invention, one polypeptide comprising, consisting essentially of, or consisting of one or more than one peptide as shown in Table 1 or equivalent thereof can be used to bind to MPO-ANCA. Alternatively, two or more, three or more, four or more, five or more, six or more, seven or more, eight or more, nine or more, ten or more, etc. polypeptides each comprising, consisting essentially of, or consisting of one or more than one peptide as shown in Table 1 or equivalent thereof can be used to bind to MPO-ANCA. In representative embodiments, each polypeptide comprises one peptide from Table 1 or an equivalent thereof and each polypeptide comprises a different peptide.

The methods of the invention can be qualitative, quantitative or semi-quantitative.

In representative embodiments, the level of binding of the polypeptide to MPO-ANCA is compared with a suitable control, e.g., a healthy subject or population of healthy subjects without an ANCA-associated disorder. In representative embodiments, the level of binding is compared with a predetermined standard (e.g., above a predetermined threshold) to determine the presence of MPO-ANCA. In particular embodiments, the subject serves as its own control. For example, levels of MPO-ANCA can be compared over time.

The predetermined standard can be determined by any suitable method known in the art. For example, the predetermined standard can be based on the level of binding in any suitable control subject or population of subjects as would be known to those skilled in the art. For example, the predetermined standard can be based on the average level of binding detected in samples from a control set of subjects. In particular embodiments, the standard is predetermined in the sense that it is fixed, for example, based on previous experience with the assay and/or a population of subjects. Alternatively, the term “predetermined standard” can also indicate that the method of arriving at the value is predetermined or fixed even if the particular value varies among assays or may even be determined for every assay run.

As used herein, a “biological sample” may comprise any suitable body fluid, tissue and/or excreta in which MPO-ANCA may be present. Suitable body fluids include, but are not limited to, lymph, blood (including whole blood, plasma [which includes plasmapheresis material], serum, and/or fractions of any of the foregoing), urine, semen, saliva, mucus, tears, sputum, bronchial secretions, joint fluid, and/or cerebrospinal fluid. Suitable tissues include, but are not limited to, spleen tissue, liver tissue, renal tissue, connective tissue, smooth muscle tissue, cardiac muscle tissue, skeletal muscle tissue, bone marrow tissue, nervous system tissue, epithelial tissue, skin, and/or lymph node. Excreta includes feces, urine, and/or sweat.

Blood samples include without limitation whole blood, plasma, serum and fractions of any of the foregoing (e.g., protein fractions and/or cellular fractions). For example, in particular embodiments, the blood sample is enriched for immunoglobulins (Ig). In representative embodiments, the blood sample is enriched for IgG, IgA and/or IgM. In embodiments of the invention, the blood sample has been processed to reduce ceruloplasmin and/or a fragment of ceruloplasmin (e.g., the 45 kilodalton cleavage product). In embodiments of the invention, the blood sample has been enriched for leukocytes, e.g., neutrophils and/or monocytes.

Any suitable method can be used to detect binding between the polypeptide and MPO-ANCA in the biological sample. In representative embodiments, the detecting step comprises an immunoassay including but not limited to immunopurification, an Enzyme Immunoassay (EIA), an Enzyme Linked-Immunosorbent assay (ELISA), Western blot and/or immunofluorescence. Numerous protocols for immunoassays are well known in the art. In certain embodiments, the immunoassays can be, e.g., immunofluorescence, immunohistochemistry, or chemiluminescence assays. Other assay techniques that can be used include, without limitation, chromatography, microscopy, mass spectroscopy, gel electrophoresis, and microarray analysis.

The polypeptide can be coupled to a solid support (e.g., beads, particles, membranes, plates, slides, chips, or wells formed from materials such as glass, latex, plastic (e.g., polystyrene, polyethylene, polypropylene), metal, rubber, or ceramic) in accordance with known techniques. Coupling to the solid support can be done by any means known in the art, such as conjugation with a coupling agent, adsorption, non-covalent interactions, covalent interactions, and electrostatic interactions. The polypeptide can be directly coupled to a detectable group or detection can proceed via a secondary reagent that specifically binds to the polypeptide. The polypeptide can be conjugated to detectable groups such as radiolabels (e.g., ³⁵S, ¹²⁵I, ¹³¹I), enzyme labels (e.g., horseradish peroxidase, alkaline phosphatase), and fluorescence labels (e.g., fluorescein, streptavidin-phycoerythrin) in accordance with known techniques. The polypeptide can also be linked indirectly to detectable groups (e.g., biotin linked to the polypeptide and streptavidin linked to the detectable group). Determination of the formation of a polypeptide/MPO-ANCA complex in the methods of this invention can be by detection of, for example, precipitation, agglutination, flocculation, radioactivity, color development or change, fluorescence, luminescence, etc., as is well-known in the art.

In representative embodiments, the detection step comprises mass spectrometry.

In other representative embodiments, the method to detect binding between the polypeptide and MPO-ANCA in the biological sample is an indirect method, e.g., that detects a change in the activity and/or function of neutrophils and/or monocytes as a result of binding between the polypeptide and MPO-ANCA, such as a neutrophil activation assay.

One aspect of the invention is a kit comprising the polypeptide of the invention. The kit can further comprise other reagents for carrying out the disclosed methods. Other reagents can include, without limitation, blocking solutions wash solutions, buffer solutions, detection molecules, controls, and standards.

Optionally, the kits can include components for carrying out assays with the additional use of detection devices for immunoassay, chemiluminescence, chromatography, spectrometry, electrophoresis, sedimentation, isoelectric focusing, or any combination thereof. Examples include, without limitation, filter plates and multi-well plates. Analysis may be carried out on a single sample or multiple samples.

In addition, the kit may optionally include instructions for performing the method or assay. Additionally, the kit may optionally include depictions or photographs that represent the appearance of positive and negative results. In some embodiments, the components of the kit may be packaged together in a common container.

The kits can include material for carrying out assays on biomarkers individually or in panels with one another or other additional biomarkers such as described herein.

Embodiments according to the present invention are described in a non-limiting examples below.

### Example 1

### Methods

### Patient Data

Chapel Hill Cohort—

Patients with ANCA small vessel vasculitis, categorized by the Chapel Hill Consensus Conference nomenclature (Jennette et al., Arthritis Rheum. 37:187 (1994)) were included in this study. The Chapel Hill cohort (Table 2) included 45 myeloperoxidase-ANCA-positive patients with MPA (37.8%), GPA (22.2%), renal-limited disease (31.1%) and overlapping pauci-immune vasculitides (2.2%). This cohort was 51.1% female and 80.3% Caucasian with a median age of 59.0 years with a range of 19-83 years. Samples for mass spectrometry studies were obtained from 52 patients with active disease and 35 patients in remission. Of these samples, there were 22 longitudinal active and remission samples from individual patients. Healthy control samples for MALDI-MS assays were from 10 volunteers who were carefully screened for autoimmune diseases, hypertension and inflammatory diseases. Sera from an additional 40 healthy volunteers were used for enzyme-linked immunosorbent assays (ELISA). Definitions of disease remission and relapse have been previously described (Nachman et al., J. Am. Soc. Nephrol. 7:33 (1996)).

Criteria for classification of ANCA-negative small vessel vasculitis required biopsy of kidney, lung or upper respiratory tract with histologic findings consistent with AAV and persistently negative ANCA tests by antigen-specific ELISA for MPO and PR3. Table 2 shows the fourteen blood samples obtained from 10 ANCA-negative patients (80% female and 80% Caucasian with a median age of 44.0 years). Three of the 10 patients had a positive p-ANCA IFA on one occasion with persistently negative MPO ELISA results. In four patients, longitudinal samples were obtained during disease activity and in remission.

ANCA-negative samples from UNC Chapel Hill were tested at the UNC Hospital clinical laboratory using both the INOVA ELISA kit and INOVA immunofluorescence. Secondly, they were assayed by an in-house ELISA (Erdbrugger et al., Kidney Int. 69:1799 (2006)). Finally, they were confirmed to be ANCA-negative by a radioimmunoassay at Massachusetts General Hospital, Boston, Mass. (Csernok et al., Rheumatology (Oxford) 43:174 (2004)). ANCA-negative samples from UMCG, Netherlands were assayed by IFA (Tervaert et al., Ann. Rheum. Dis. 52:115 (1993)) and in-house capture ELISA (Savige et al., Am. J. Clin. Pathol. 120:312 (2003)).

TABLE 2
Patient Demographics

| | MPO-ANCA | MPO-ANCA | ANCA-Negative | ANCA-Negative | Healthy | Healthy |
|---|---|---|---|---|---|---|
| | Cohort (CH) | Cohort (NL) | Cohort (CH) | Cohort (NL) | Volunteers (CH) | Volunteers (NL) |
| Median age (range) | 59.0 (19-83) | 66.0 (41-78) | 44.0 (12-72) | 60.7 (17-69) | 27.5 (20-53) | 35.0 (25-59) |
| Sex | | | | | | |
| Male | 48.9% (22/45) | 50.0% (10/20) | 20.0% (2/10) | 38.5% (5/13) | 40.0% (4/10) | 55.6% (5/9) |
| Female | 51.1% (23/45) | 50.0% (10/20) | 80.0% (8/10) | 61.5% (8/13) | 60.0% (6/10) | 44.4% (4/9) |
| Race | | | | | | |
| Asian | 0.0% (0/45) | 0.0% (0/20) | 0.0% (0/10) | 0.0% (0/13) | 0.0% (0/10) | 0.0% (0/9) |
| African American | 13.3% (6/45) | 0.0% (0/20) | 10.0% (1/10) | 0.0% (0/13) | 10.0% (1/10) | 0.0% (0/9) |
| Hispanic | 4.4% (2/45) | 0.0% (0/20) | 0% (0/10) | 0.0% (0/13) | 0.0% (0/10) | 0.0% (0/9) |
| Caucasian | 80.3% (36/45) | 95.0% (19/20) | 80.0% (8/10) | 100.0% (13/13) | 70.0% (7/10) | 100% (9/9) |
| Other | 1.5% (1/45) | 5.0% (1/20) | 10.0% (1/10) | 0.0% (0/13) | 20.0% (2/10) | 0.0% (0/9) |
| Diagnosis | | | | | | |
| MPA | 37.8% (17/45) | 45.0% (9/20) | 30.0% (3/10) | 0.0% (0/13) | — | — |
| Renal Limited | 31.1% (14/45) | 40.0% (8/20) | 0.0% (0/10) | 38.5% (5/13) | — | — |
| GPA | 22.2% (10/45) | 15.0% (3/20) | 60.0% (6/10) | 61.5% (8/13) | — | — |
| Churg-Strauss | 6.7% (3/45) | 0.0% (0/20) | 0.0% (0/10) | 0.0% (0/13) | — | — |
| Autoimmune | 2.2% (1/45) | 0.0% (0/20) | 0.0% (0/10) | 0.0% (0/13) | — | — |
| Overlap | | | | | | |
| Unknown | 0.0% (0/45) | 0.0% (0/20) | 10.0% (1/10) | 0.0% (0/13) | — | — |
| Disease Activity | | | | | | |
| Active Disease | 60.0% (52/87 | 50.0% (20/40 | 71.4% 10/14 | 50.0% (11/22 | — | — |
| (BVAS > 0) | samples) | samples) | samples) | samples) | | |
| Clinical Remission | 40.0% (35/87 | 50.0% (20/40 | 28.6% (4/14 | 50.0% 11/22 | — | — |
| (BVAS = 0) | samples) | samples) | samples) | samples) | | |
| Organ Involvement | | | | | | |
| Lung | 44.4% (20/45) | 25.0% (5/20) | 20.0% (2/10) | 23.1% (3/13) | — | — |
| Upper Resp. | 26.7% (12/45) | 25.0% (5/20) | 50.0% (5/10) | 61.5% (8/13) | — | — |
| Joints | 46.7% (21/45) | 15.0% (3/20) | 60.0% (6/10) | 38.5% (5/13) | — | — |
| GI | 4.4% (2/45) | 0.0% (0/20) | 0.0% (0/10) | 0.0% (0/13) | — | — |
| Muscles | 0.0% (0/45) | 5.0% (1/20) | 0.0% (0/10) | 0.0% (0/13) | — | — |
| Neuro | 4.4% (2/45) | 15.0% (3/20 ) | 10.0% (1/10) | 7.7% (1/13) | — | — |
| Derm | 8.9% (4/45) | 15.0% (3/20) | 30.0% (3/10) | 7.7% (1/13) | — | — |
| Kidney | 95.6% (43/45) | 85.0% (17/20) | 40.0% (4/10) | 46.2% (6/13) | — | — |
| Outcomes | | | | | | |
| Death | 6.7% (3/45) | 0.0% (0/20) | 0.0% (0/10) | 0.0% (0/13) | — | — |
| ESRD | 15.6% (7/45) | 30.0% (6/20) | 10.0% (1/10) | 7.7% (1/13) | — | — |
| Number of Relapses | | | | | | |
| 0 | 53.3% (24/45) | 95.0% (19/20) | 30.0% (3/10) | 92.3% (12/13) | — | — |
| 1 | 24.4% (11/45) | 5.0% (1/20) | 20.0% (2/10) | 7.7% (1/13) | — | — |
| 2 | 15.5% (7/45) | 0.0% (0/20) | 30.0% (3/10) | 0.0% (0/13) | — | — |
| 3 | 2.2% (1/45) | 0.0% (0/20) | 10.0% (1/10) | 0.0% (0/13) | — | — |
| 4 | 4.4% (2/45) | 0.0% (0/20) | 0.0% (0/10) | 0.0% (0/13) | — | — |
| 5 | 0.0% (0/45) | 0.0% (0/20) | 10.0% (1/10) | 0.0% (0/13) | — | — |
| Average number of | 6.0 ± 5.98 | 3.1 ± 2.2 | 2.6 ± 4.97 | 7.3 ± 6.6 | — | — |
| months Cytoxan (IV | | | | | | |
| or PO) | | | | | | |
| Average number of | 23.5 ± 31.71 | 5.7 ± 3.5 | 28.3 ± 19.64 | 7.5 ± 6.5 | — | — |
| months consecutive | | | | | | |
| immunosuppressives | | | | | | |

The potential that medications may influence autoantibody detection was examined. There was no obvious bias. The patient cohort was inclusive of all stages of disease with varied treatment regimens. The study included 11/52 samples collected at disease onset prior to initiation of treatment. Of the 35 patients in remission, 11 remained positive for anti-MPO reactivity. Three were on no immunosuppressive or maintenance therapy and the remainder were on a variety of therapy including: prednisone, methylprednisolone, azathioprine and mycophenolate mofetil (Table 2).

Groningen, The Netherlands Replication Study—

An independent replication cohort was conducted in Groningen, The Netherlands (demographics shown in Table 1). Immunoglobulin samples (n=49) obtained from the University Medical Center Groningen (UMCG) included MPO-ANCA glomerulonephritis patients with active disease (n=20) and in disease remission (n=20), in addition to nine samples from healthy individuals. The 49 samples included patients with MPA (52.2%), renal-limited disease (34.8%), and GPA (13.0%). This cohort was 47.8% female and 95.7% Caucasian with a mean age of 61.9 years.

UMCG had an ANCA-negative cohort (Table 2) of 13 unique individuals (one individual was positive for elastase) with samples from active disease (n=11) and disease remission (n=11) included patients with GPA (61.5%) and renal-limited disease (38.5%). This cohort was 61.5% female and 100% Caucasian with a median age of 60.7 years. Matched, active disease versus remission, samples were obtained from nine patients. At UMCG, criteria for ANCA negative SVV required kidney, lung, or nose biopsies showing pauci-immune extracapillary glomerulonephritis with or without fibrinoid necrosis and crescents in the kidney (n=5), capillaritis or granuloma of the lung (n=1) or upper respiratory tract (n=7) with pathologic evidence of necrotizing vasculitis or leukocytoclastic vasculitis. Sera were routinely tested by indirect immunofluorescence by the laboratory of Clinical Immunology UMCG as described (Tervaert et al., Ann. Rheum. Des. 52:115 (1993)) and was found negative in 10 of 13 patients in the ANCA negative cohort at the moment of diagnosis (the 3 sera positive on IIF showed an atypical (n=2) or perinuclear (n=1) pattern of fluorescence). All sera were also tested in an in-house developed capture ELISA for antibodies against Proteinase 3, and myeloperoxidase performed at the laboratory of Clinical Immunology UMCG and determined to be negative on all ANCA negative patients (Savige et al., Am. J. Clin. Pathol. 120:312 (2003)). One patient was found positive for antibodies against elastase by capture ELISA.

### Matrix-Assisted Laser Desorption Ionization Time of Flight Mass Spectrometry (MALDI-TOF/TOF MS)

Total immunoglobulin (IgG) was purified from sera using protein A/G PLUS-Agarose Reagent according to commercial protocol (Santa Cruz Biotechnology). Purified IgG was immobilized on CNBr-activated Sepharose 4B (GE Healthcare) in compact reaction columns (CRC, USB Corporation) and exposed to native MPO protein (MPO, Elastin Products Co, Inc) followed by digestion with sequencing grade TPCK-treated trypsin (Worthington). MPO-peptides remaining bound to IgG after digestion were eluted with 0.1% trifluoro acetic acid and analyzed on a 4800 Plus Matrix-Assisted Laser Desorption Ionization Time of Flight Mass Spectrometry (MALDI TOF/TOF MS/MS) in conjunction with ProteinPilot software (AB SCIEX).

Mass spectrometry analysis of ANCA-negative IgG and mouse IgG samples were conducted as above. With the exception of protein substrate, immobilized ANCA-negative IgG were exposed to whole leukocyte lysate preparation from a healthy donor and mouse IgG to recombinant mouse MPO (R&D Systems).

To identify epitopes of low titer autoantibodies, ¹⁸O labeling (Mirza et al., J. Proteome Res. 7:3042 (2008)) was used to differentiate between the signal and the noise in the MS and MSMS spectra using the 4 Da shifts between the corresponding spectra of labeled and non-labeled peptides. Mass spectrometry analysis for epitopes recognized by MPO autoantibodies from healthy individuals, and for detection of low ANCA titers in remission samples required the use of isotope H₂¹⁸O to increase sensitivity (Cambridge Isotope Laboratories). Samples from healthy individuals (n=10) and MPO-ANCA patients in clinical remission (n=35) were processed in the presence of H₂¹⁸O. Incorporation of H₂¹⁸O on the carboxyl terminus of each proteolytic fragment of MPO during tryptic digestion, trypsin buffer was made with the isotope H₂¹⁸O as a diluent.

To identify linear epitopes versus conformational epitopes, native MPO was predigested/fragmented with immobilized trypsin (Promega) prior to exposure to Ig fractions. Positive identification of a bound fragment by mass spectrometry indicated a linear epitope.

### Enzyme-Linked Immunosorbent Assay (ELISA)

Chapel Hill—

A total of 195 IgG samples were tested by native MPO or peptide ELISA. Samples included those from healthy controls (n=59), active disease (n=80) and remission patients (n=56). Total IgG was added to wells (2 μg/well) precoated with 1-2 μg peptide (UNC-CH peptide synthesis core). Goat anti-human with alkaline phosphatase conjugate secondary antibody specific to IgG (H+L) (Millipore, Billerica, Mass.) (1:10,000) was added and detected using 1-Step PNPP substrate (Thermo Scientific) and read at λ=405 nm after 30 min.

Groningen, The Netherlands (NL)—

Identical peptides were used in ELISAs at both locations; all other substrates used to test NL cohort samples by ELISAs were prepared in Groningen. Total IgG (1:500˜2 mg/well) was added to precoated wells blocked with 1% BSA. Secondary antibody, goat anti-human conjugated with alkaline phosphatase (A5403; Sigma-Aldrich) (1:5000), was added and detected using p-nitrophenyl-phosphate disodium substrate.

### Identification of the Epitope-Masking Factor in Sera

Serum proteins that bound immobilized MPO peptide aa447-459 were eluted and sequenced for identification. A protein present in both patient and healthy sera was observed at ˜50 kD by SDS-PAGE. Sequenced on a 4800 MALDI TOF-TOF, the protein was identified as ceruloplasmin (CP), and confirmed by western blot analysis probed with a rabbit anti-human anti-CP polyclonal antibody (Abeam). Native CP was purchased (Enzo Life Science) and proteolytically digested in vitro by plasmin (Haemtech).

### In Vitro Neutrophil Activation

Neutrophil activation assays were performed using MPO-ANCA affinity-purified to linear MPO epitopes. Reactivity and specificity of purified antibodies were confirmed by ELISA. Human neutrophils isolated from four healthy donors were purified (Falk et al., Proc. Natl. Acad. Sci. USA 87:4115 (1990)) (2), and primed with cytochalasin B (Sigma-Aldrich). Activation of human neutrophils was assessed by the amount of reactive oxygen species released using the superoxide dismutase.

### In Vivo Assay

Actively Induced Anti-MPO Associated Glomerulonephritis—

DR2 Tg mice (Ooi et al., Proc. Natl. Acad. Sci. USA 109:E2615 (2012); Rich et al., Eur. J. Immunol. 34:1251 (2004)) were immunized s.c. with 3×100 μg of peptide antigen (OVA₃₂₃₋₃₃₉, n=4 or MPO₄₄₂₋₄₆₀, n=10) first in FCA then FIA on days 0, 7 and 14. Mice were culled on day 28.

MPO-ANCA Induced Glomerulonephritis—

Serum IgG purified from DR2 Tg mice immunized with either OVA₃₂₃₋₃₃₉ or MPO₄₄₂₋₄₆₀ were passively transferred i.v. (35 μg/g, n=5 per group) into LPS primed (1 μg/g i.p.) DR2 Tg naïve recipient mice, that were culled 6 days later.

Assessment of Injury—

For indirect immunofluorescence, ethanol fixed thioglycolate induced peritoneal neutrophils were cytospun onto slides, purified serum IgG (1 mg/ml) was applied for 20 minutes then anti-mouse IgG Ab was detected using FITC conjugated anti-mouse IgG Ab (Silenus) (Ooi et al., Proc. Natl. Acad. Sci. USA 109:E2615 (2012); Lock, J. Clin. Pathol. 47:4 (1994)). Albuminuria was assessed by ELISA (Bethyl Laboratories) and haematuria assessed by urine test strips (Combur) from a 24 hour urine sample. Blood urea nitrogen (BUN) was measured using standard laboratory methods on serum collected at the end of experiment. Glomerular abnormalities assessed, on 3 gam thick, PAS stained, formalin fixed, paraffin embedded sections (≧50 glomeruli/mouse), were thickening of the capillary walls and mesangial hypercellularity (Ooi et al., J. Am. Soc. Nephrol. 20:980 (2009)). Total glomerular cell nuclei were enumerated (≧20 glomeruli/mouse). Glomerular neutrophils were detected by immunoperoxidase staining of 6 μm thick, periodate lysine paraformaldehyde fixed, frozen kidney sections using anti-Gr-1 antibodies (RB6-8C5).

### Statistical Analysis

P values were calculated by Wilcoxon two sample tests for two samples comparisons, Kruskal-Wallis Test for three-groups comparison and Signed Rank Test for paired groups comparisons. Bonferroni correction was used, α=0.05/3=0.167. For in vivo mouse studies, P values were calculated using student's t-test.

### Example 2

### Epitope Diversity in Patients with MPO-ANCA

MPO-ANCA positive patients (n=45) from the University of North Carolina cohort (UNC) were analyzed in the initial studies. Demographics of the cohort (Table 2) indicate a predominantly Caucasian group. Approximately 40% are diagnosed with microscopic polyangiitis (MPA), 40% with renal-limited disease and 20% are diagnosed with granulomatosis with polyangiitis (GPA). To compare epitope specificities related to disease activity, longitudinal samples were analyzed to include active disease (n=52) and remission (n=35) from the UNC cohort (Tables 3 and 4). Ten healthy subjects were included in the analyses. Purified immunoglobulin (Ig) from sera samples was subjected to analysis by epitope excision MALDI-TOF/TOF mass spectrometry (depicted in FIG. 3A) to identify epitope profiles of autoantibodies reactive with MPO. In order to epitope map low titer anti-MPO antibodies from healthy subjects and from patients in disease remission, an approach was designed using the ¹⁶O-to-¹⁸O exchange technique, an adaptation of a proteomics-based quantification method (Mirza et al., J. Proteome Res. 7:3042 (2008)). Incorporation of H₂¹⁸O on the carboxyl termini of proteolytic fragments produces a 4 Da mass shift in the MS spectra, decipherable from background noise. MPO epitopes unique to Ig from patients with active disease were designated ‘exclusive to active disease’; epitopes identified in remission samples were designated ‘persistent during remission’; and those present in samples from healthy subjects were designated asymptomatic or ‘natural’.

TABLE 3
MPO-ANCA Cohort (CH): Description of 87 sera samples

| | Number | Mean age ± std (years) | % Female |
|---|---|---|---|
| Clinical Remission | 35 | 61.6 ± 16.8 | 42.9% (15/35) |
| Active Disease: | 52 | 53.7 ± 18.8 | 50% (26/52) |
| Onset of disease | 22 | 55.8 ± 18.5 | 50% (11/22) |
| Relapse | 14 | 61.9 ± 15.6 | 35.7% (5/14) |
| Smoldering Active | 8 | 53.1 ± 13.1 | 87.5% (7/8) |
| Remission on | 5 | 70.4 ± 9.3 | 20% (1/5) |
| Therapy | | | |
| Unresponsive to | 3 | 54.0 ± 21.0 | 66.7% (2/3) |
| treatment | | | |

TABLE 4
MPO-ANCA Cohort (NL): Description of 40 sera samples

| | Number | Mean age ± std (years) | % Female |
|---|---|---|---|
| Clinical Remission | 20 | 66.2 [41.7-78.4] | 50% (10/20) |
| Active Disease: | 20 | 65.5 [41.5-77.9] | 50% (10/20) |
| Onset of disease | 20 | 65.5 [41.5-77.9] | 50% (10/20) |
| Relapse | | | |
| Smoldering Active | | | |
| Remission on Therapy | | | |
| Unresponsive to | | | |
| treatment | | | |

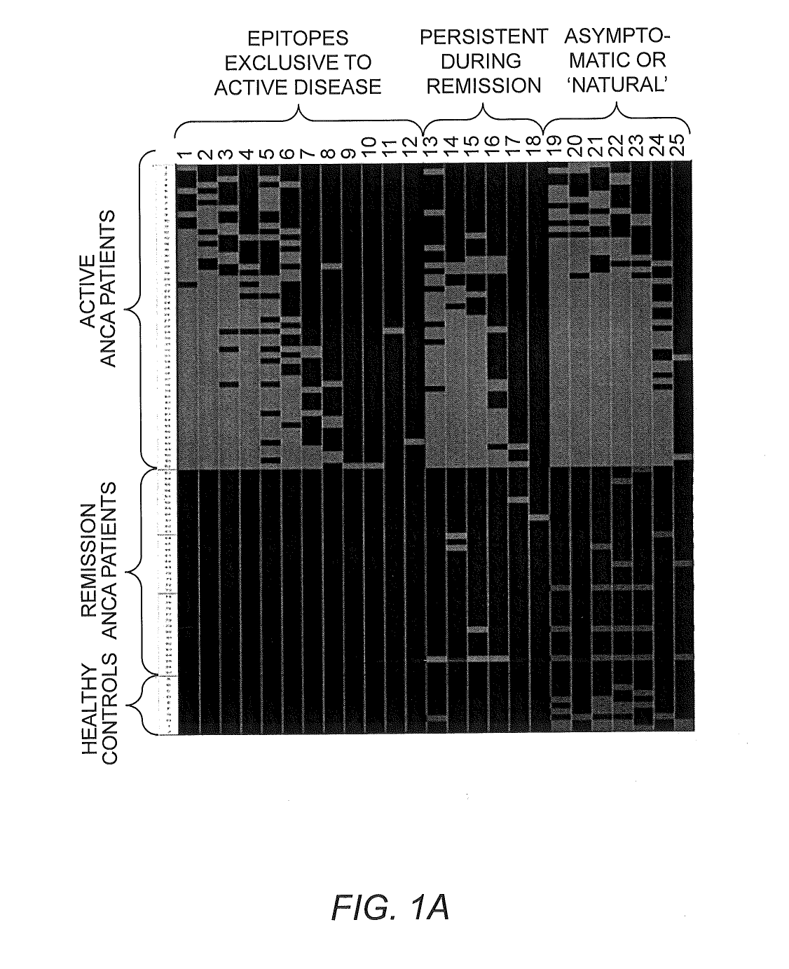

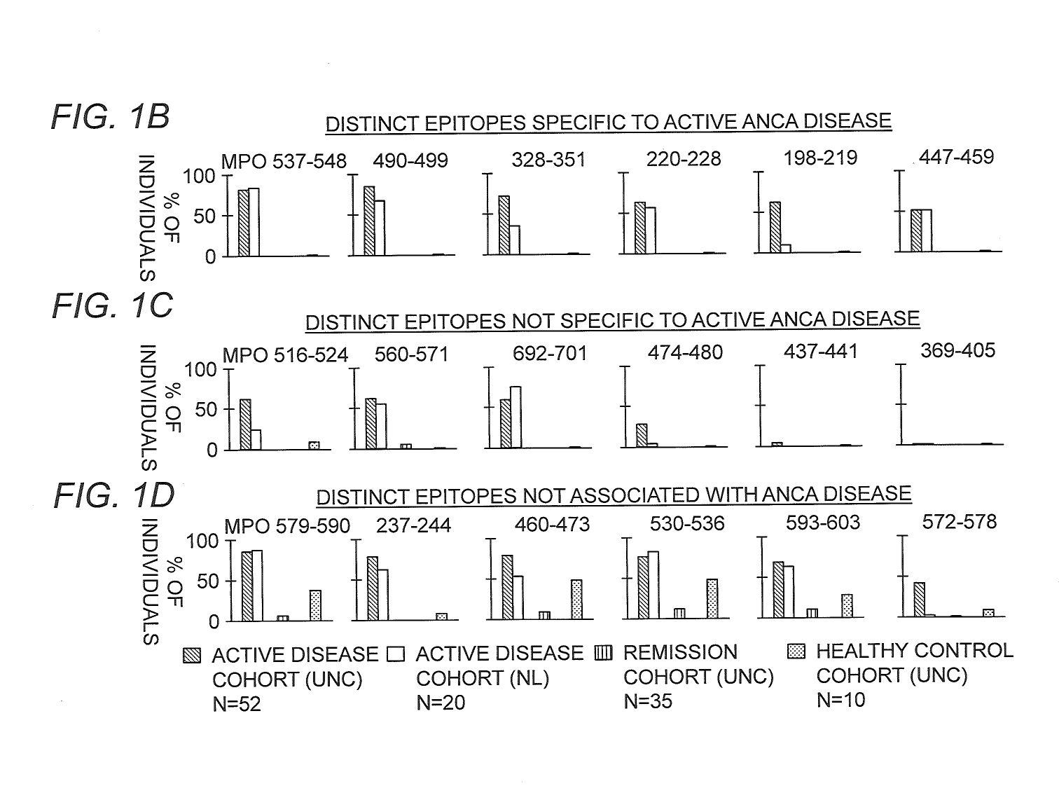

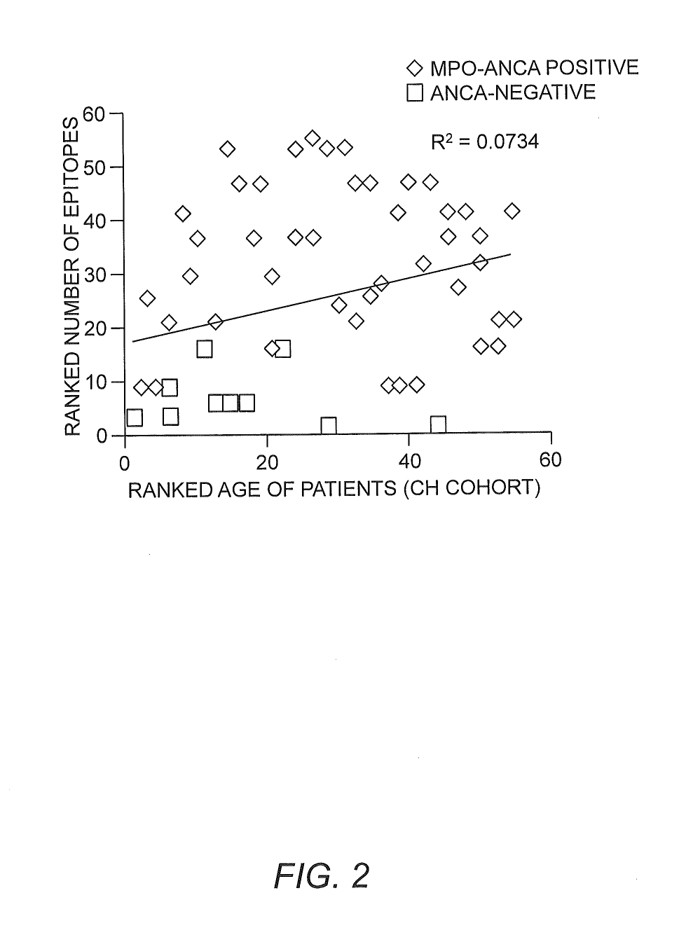

A total of 25 anti-MPO autoantibody epitopes were identified and categorized by disease association (FIG. 1A). Twelve epitopes were unique and exclusive to MPO-ANCA expressed during active disease. Eight epitopes were reactive with Ig from healthy subjects. Notably, asymptomatic or ‘natural’, autoantibodies were present in all active disease samples. There was no correlation between number of epitope-specific autoantibodies and age of the individual at the time of the sample (R²=0.074) (FIG. 2).

To examine whether the active disease MPO-ANCA epitope profile was universal to ANCA disease, a replication study was performed on a cohort from Groningen, The Netherlands (NL) of patients with active disease (n=20) (Table 2). The frequencies of the six most prominent epitopes within each disease classification are shown in FIGS. 1B-1D. The data indicate the frequencies of specific epitopes, comparing UNC and NL cohorts. MPO-ANCA epitopes found in both the UNC and NL cohorts were consistent in specificity, although they differed in distribution.

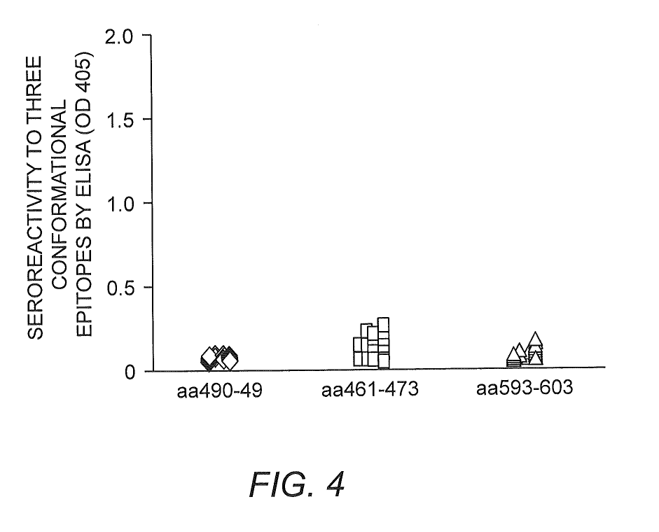

Both conformational and linear epitopes are detected by epitope excision MALDI-TOF/TOF MALDI-MS. To distinguish conformational epitopes from linear epitopes, the protocol was modified such that Ig was incubated with pre-digested MPO protein with the assumption that loss of epitope reactivity signified a dependence on conformation. Of the 25 epitopes originally identified, 20 were conformational. Synthetic peptides corresponding to the five linear peptides were tested by ELISA for validation of MALDI-MS results. A linear epitope located on the heavy chain of MPO (aa447-459), which was exclusively associated with active disease by MS, was confirmed by ELISA with reactivity in 42.9% (21/49) of UNC patient samples and in 52.2% (12/23) of the NL population (FIG. 3A). Reactivity declined in clinical remission for both the UNC and NL cohorts (FIG. 3A). Of note, all samples with borderline or low optical density (OD) values by ELISA, which had not been tested previously by MS, were analyzed to determine whether they were negative for the epitope or whether they were low-titer positive. MPO-ANCA patients with values in the range of healthy individuals were found to be negative by MS analysis. MPO peptide (aa516-524), a second linear epitope identified by MS, was confirmed by ELISA to be reactive with Ig from active disease samples; however, reactivity was found in remission samples in both cohorts (FIG. 3B). In an effort to validate conformational MPO epitopes, peptides of the amino acid sequences were synthesized and used as substrates for ELISAs. Ig samples positive by MS were negative by ELISA confirming that these are conformationally-dependent epitopes (FIG. 4).

Intriguingly, placement of epitopes on the crystal structure of MPO as a monomer, shown in FIG. 3C, revealed that epitopes of asymptomatic or ‘natural’ autoantibodies are spatially adjacent to epitopes ‘exclusive to active disease’. Essential amino acids within these epitopes, in bold, were identified by fine epitope mapping using a chymotrypsin enzymatic digestion (Table 5). The sequential alignment of epitopes is suggestive of epitope spreading from non-pathogenic to pathogenic epitopes as disease develops.

TABLE 5

| Trypsin Cut (RK) | Chymotrypsin Cut (FYWLM) |
|---|---|
| NGFPVALAR | NGFPVALAR |
| (SEQ ID NO: 4) | (SEQ ID NO: 4) |
| FPTDQLTPDQER | FPTDQLTPDQER |
| (SEQ ID NO: 21) | (SEQ ID NO: 21) |
| FQDNGR | FQDNGR |
| (SEQ ID NO: 7) | (SEQ ID NO: 7) |
| RKIVGAMVQIITYR | RKIVGAMVQIITYR |
| (SEQ ID NO: 32) | (SEQ ID NO: 32) |
| IANVFTNAFR | IANVFTNAFR |
| (SEQ ID NO: 1) | (SEQ ID NO: 1) |
| VFFASWR | VFFASWR |
| (SEQ ID NO: 20) | (SEQ ID NO: 20) |
| QNQIAVDEIR | QNQIAVDEIR |
| (SEQ ID NO: 13) | (SEQ ID NO: 13) |
| IGLDLPALNMQR | IGLDLPALNMQR |
| (SEQ ID NO: 19) | (SEQ ID NO: 19) |
| QALAQISLPR | QALAQISLPR |
| (SEQ ID NO: 14) | (SEQ ID NO: 14) |
| YQPMEPNPR | YQPMEPNPR |
| (SEQ ID NO: 18) | (SEQ ID NO: 18) |
| DHGLPGYNAWR | DHGLPGYNAWR |
| (SEQ ID NO: 23) | (SEQ ID NO: 23) |
| DYLPLVLGPTAMR | DYLPLVLGPTAMR |
| (SEQ ID NO: 33) | (SEQ ID NO: 33) |

### Example 3

### “ANCA-Negative” AAV Patients have MPO-ANCA Specific for an Epitope Exclusive to Active Disease

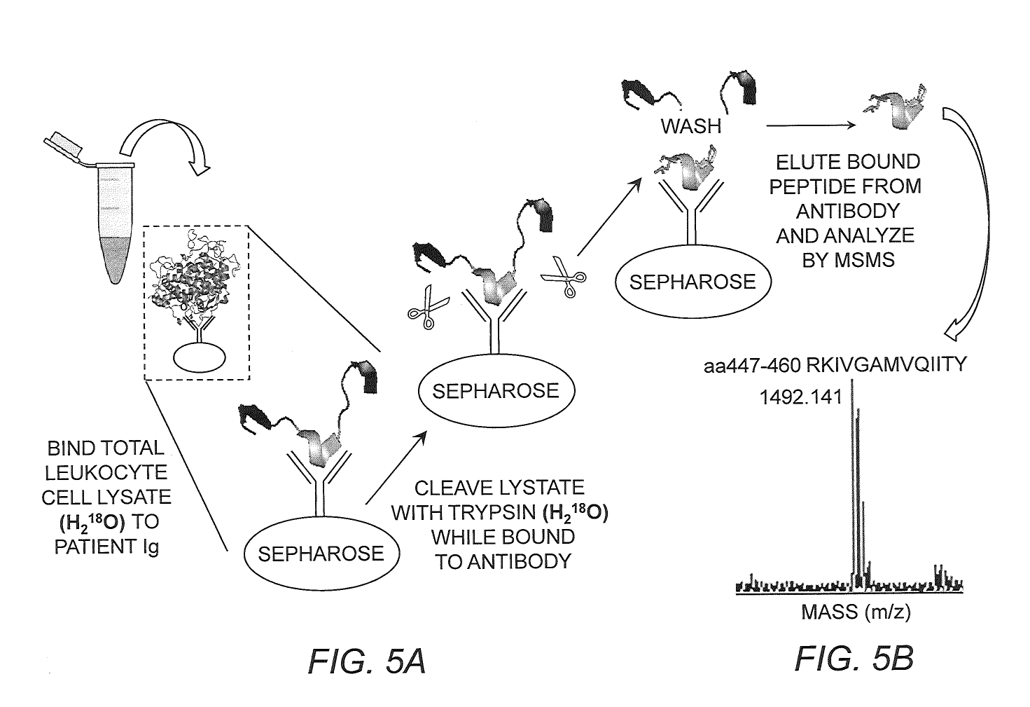

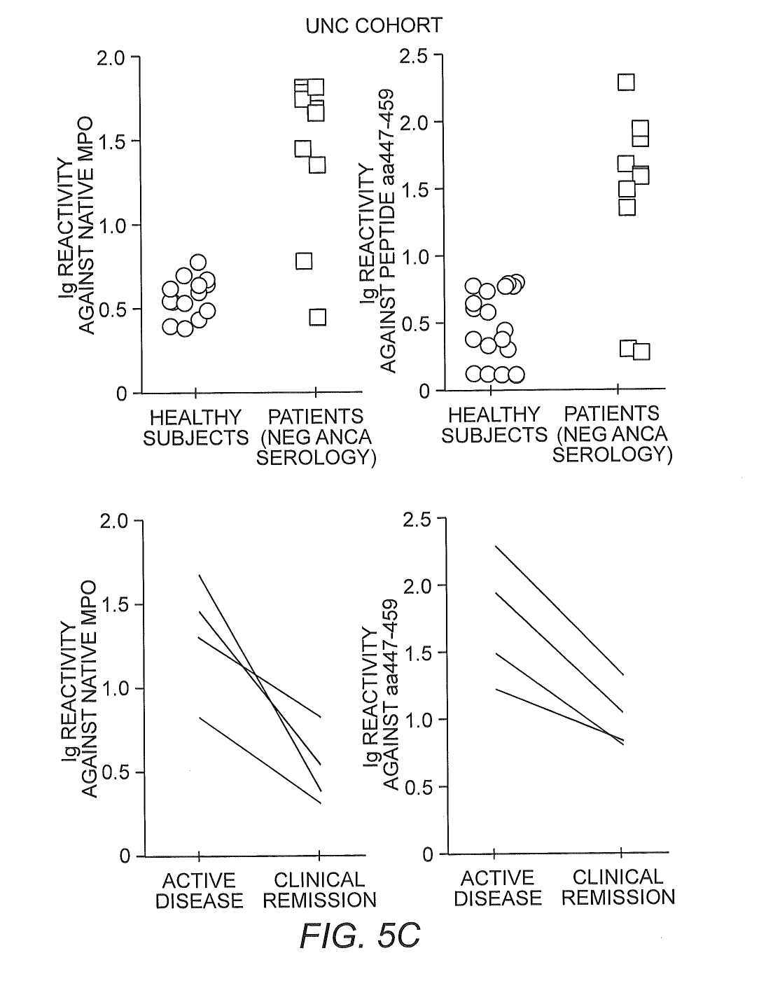

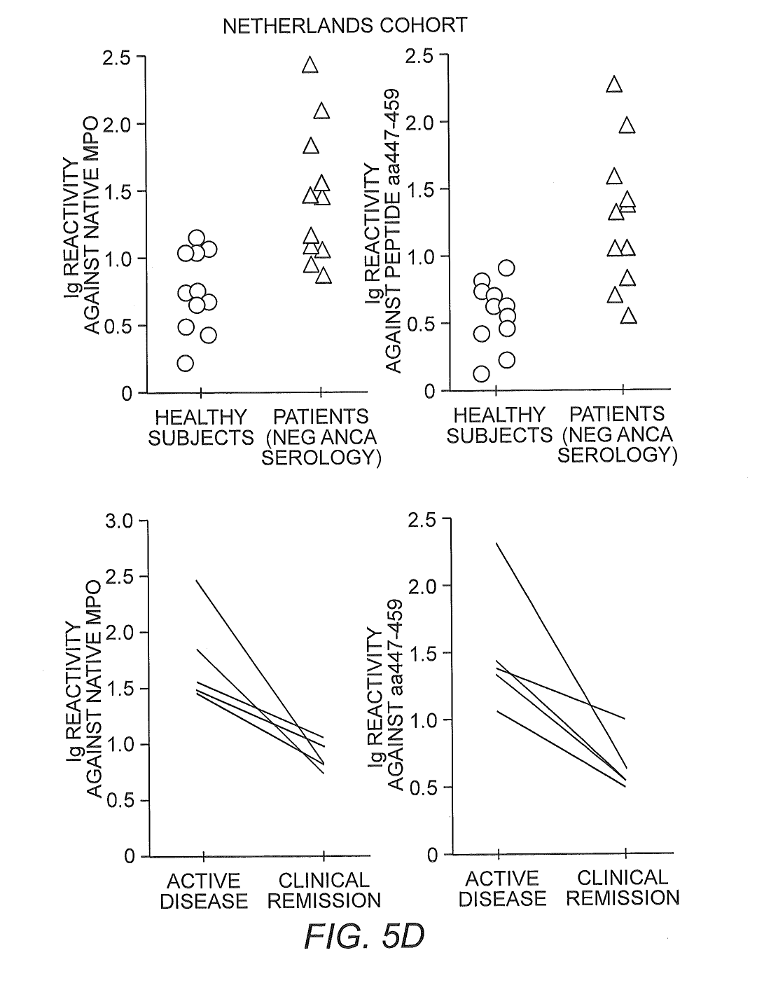

A variation of epitope excision MS for MPO-ANCA was adapted into a ‘discovery’ tool for identifying unknown autoantigens in ANCA-negative AAV, which presents clinically as vasculitis although with a negative serology for MPO-ANCA and PR3-ANCA. ANCA-negative AAV patients were identified by retrospective review of the medical records, with criteria of histological evidence of vasculitis and negative serological test results from the UNC Hospital clinical laboratory. As an additional measure, sera samples selected for analysis were retested for ANCA reactivity by the laboratory of Dr. John Niles, Massachusetts General Hospital, Boston, USA and were confirmed to be ANCA-negative. Protein lysate of total peripheral blood leukocytes were probed for antigen/s that were reactive with purified total Ig from ANCA-negative AAV patients' sera (FIG. 5A). A sole peptide was eluted and identified in the MS spectrum analysis at a mass of 1492.141 (FIG. 5B), which corresponded to the MPO peptide aa447-459 (RKIVGAMVQIITY (SEQ ID NO: 6)), the linear epitope exclusive to active disease identified in ˜50% of MPO-ANCA positive AAV patients with active disease. Unexpectedly, purified Ig from these patients reacted with native MPO by ELISA even though sera from these patients did not (FIGS. 5C and 5D). MPO-ANCA specific for this epitope were detected by ELISA using isolated Ig from 8/10 UNC AAV patients and from 6/11 NL AAV patients (Table 2). Levels of reactivity to native MPO and aa447-459 peptide corresponded well with disease activity, declining to low levels as patients entered disease remission in both the UNC (n=4) and NL (n=5) patient cohorts (Table 2).

### Example 3

### An Epitope-Masking Factor in Sera Interferes with Conventional Serologic Testing for MPO-ANCA in ANCA-Negative AAV

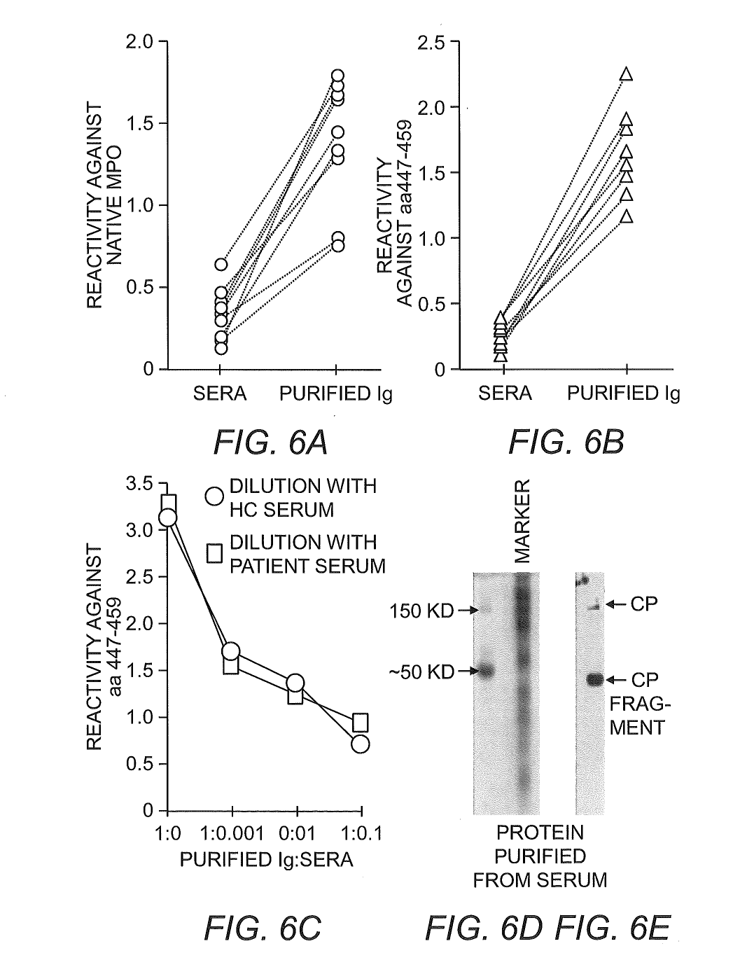

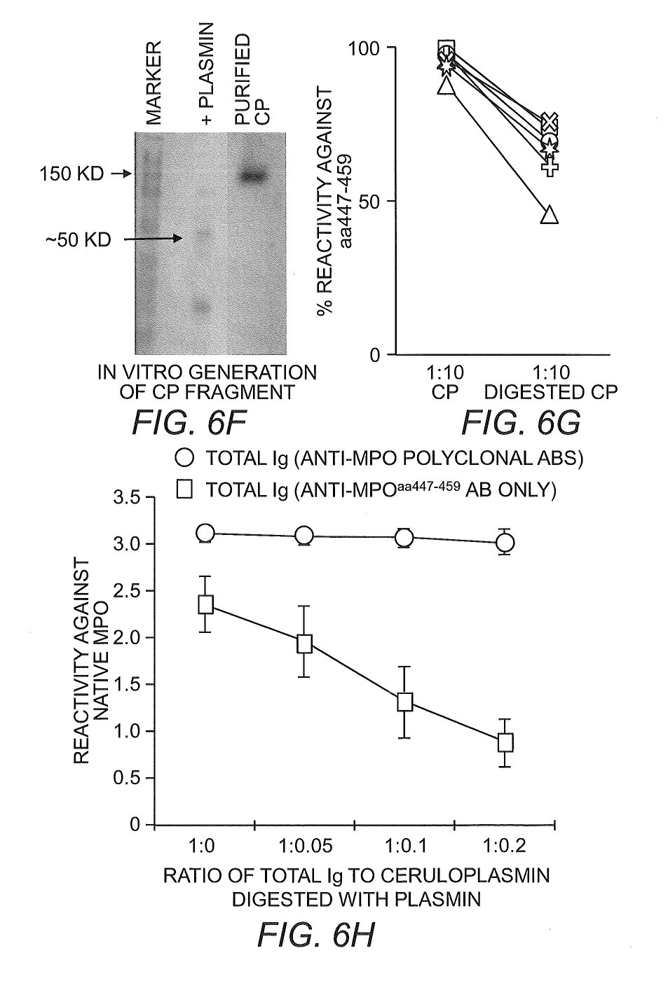

Reevaluation of ANCA-negative patients' sera by directly comparing purified Ig from each patient confirmed that anti-MPO⁴⁴⁷⁻⁴⁵⁹ autoantibodies cannot be detected by conventional serologic tests, but purified Ig readily detect substrates of both native MPO protein (FIG. 6A) and MPO aa447-459 peptide (FIG. 6B). Reactivity to either substrate was blocked when limited dilutions of sera from either a patient with ANCA disease or from a healthy subject were serially added back to patient Ig (FIG. 6C). We hypothesized that a component in sera was obscuring detection of antibodies reactive with MPO aa447-459.

To isolate the putative serum factor, immobilized MPO aa447-459 peptide was used as bait to fish-out host proteins with binding capacity. Ceruloplasmin (CP) was identified by mass spectrometry as a binding partner. However, the CP bound to aa447-459 was primarily a 50 kD protein on sodium dodecyl sulfate polyacrylamide gel electrophoresis (SDS-PAGE), while full length CP is 151 kD (FIG. 6D). An anti-ceruloplasmin western blot confirmed the fragment to be a portion of CP (FIG. 6E). To test the role of proteolytic cleavage in generating the blocking factor, a plasmin-generated fragment of CP (Kingston et al., Proc. Natl. Acad. Sci. USA 74:5377 (1977)) was tested for epitope masking capability (FIG. 6F). The digested CP decreased anti-MPO⁴⁴⁷⁻⁴⁵⁹ autoantibody reactivity by 30-50% (n=8) (FIG. 6G), while full length CP did not (FIG. 6G). The effect of the CP masking fragment was selective for blocking anti-MPO⁴⁴⁷⁻⁴⁵⁹ reactivity and did not affect the overall binding of polyclonal MPO-ANCA Ig to native MPO (FIG. 6H).

### Example 4

### Pathogenic Potential of Anti-MPO Autoantibodies Specific for MPO Epitope Aa447-459

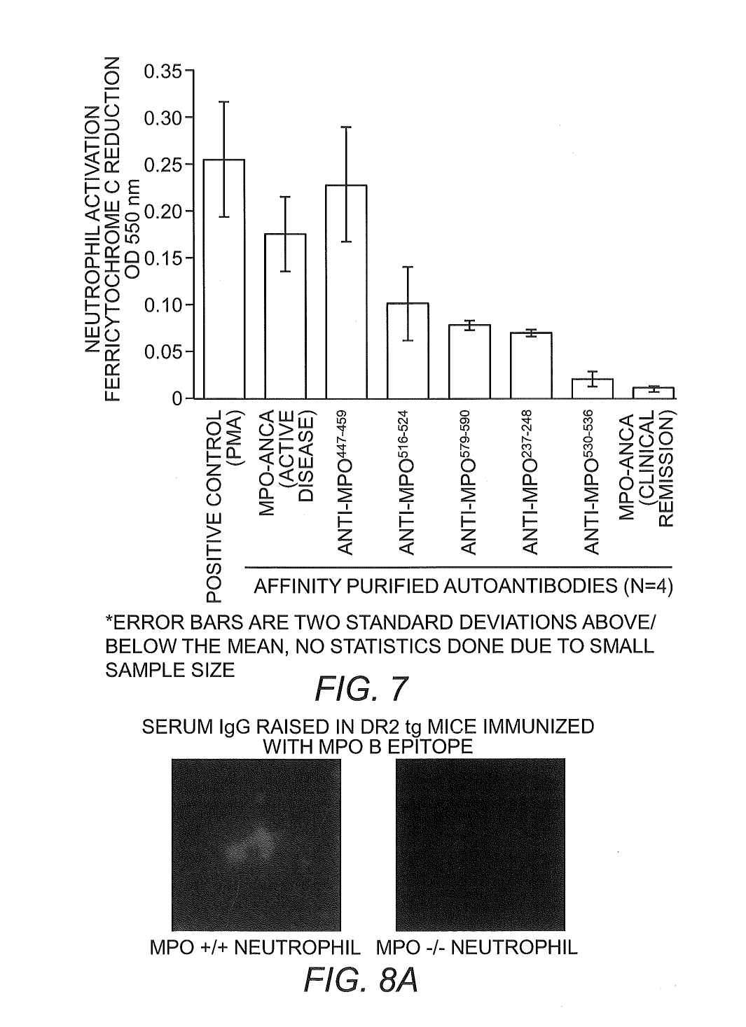

Anti-MPO⁴⁴⁷⁻⁴⁵⁹ autoantibodies induced neutrophilic release of reactive oxygen species similar to total MPO-ANCA in an in vitro activation assay (FIG. 7), indicative of a pathogenic potential. In comparison, three asymptomatic or ‘natural’ anti-MPO-autoantibodies purified from patients' sera (n=4) during disease remission, and adjusted for concentration, showed essentially no release of reactive oxygen species. This study replicates prior studies of MPO-ANCA activation of neutrophils (Falk et al., Proc. Natl. Acad. Sci. USA 87:4115 (1990)).

In collaboration with colleagues at Monash University, the pathogenic potential of anti-MPO autoantibodies targeting epitope aa447-459 was studied in their established animal model. Mice deficient in murine MHC II and transgenic for HLA-DRB1*15:01 (DR2 Tg mice) were immunized with murine MPO peptide aa409-428 (PRWNGEKLYQEARKIVGAMV (SEQ ID NO: 34), an epitope that induces glomerulonephritis in mice (Ooi et al., Proc. Natl. Acad. Sci. USA 109:E2615 (2012)). This pathogenic murine T cell epitope overlaps with the human disease-associated B cell epitope aa447-459 (RIUVGAMVQIITY (SEQ ID NO: 6)).

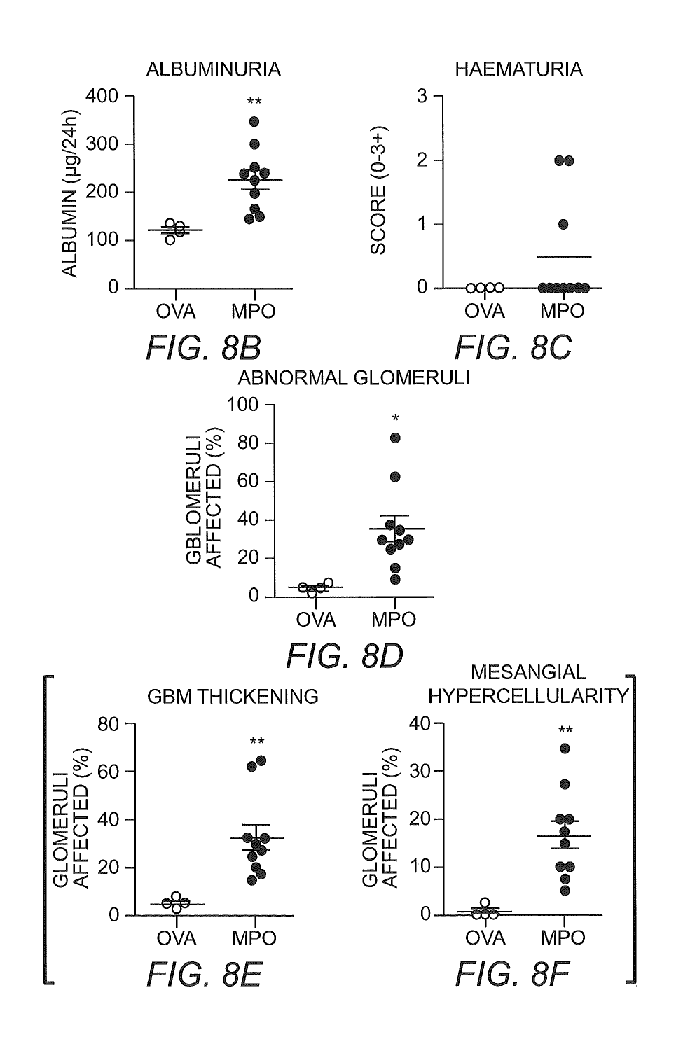

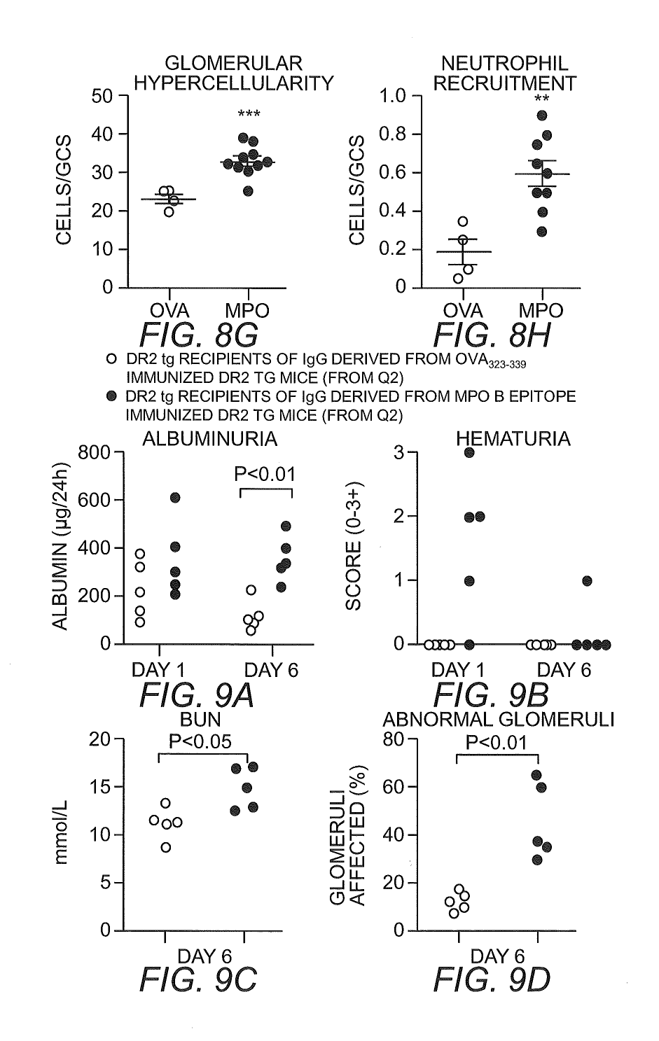

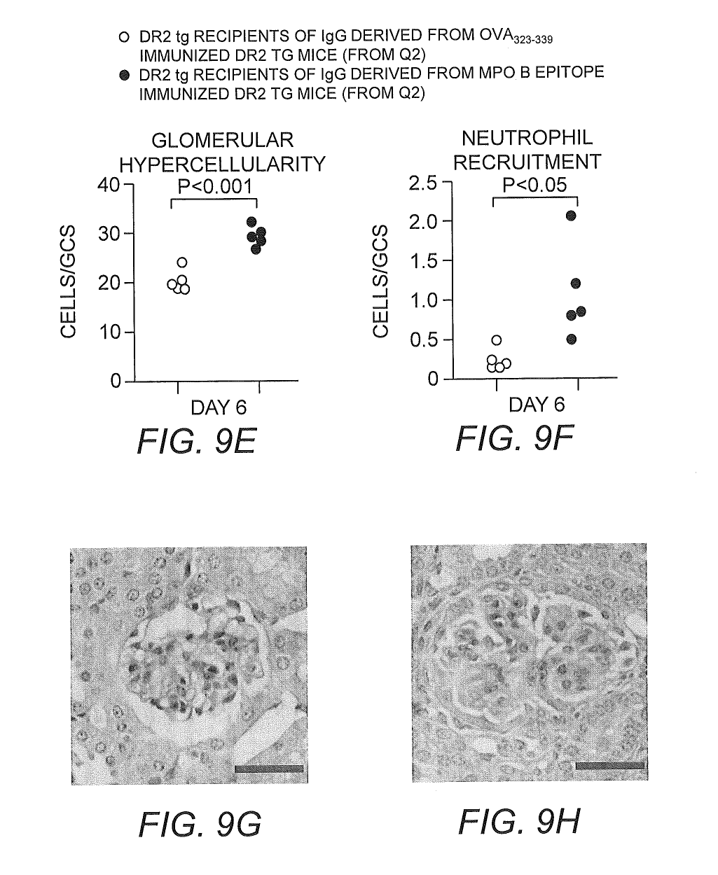

DR2 Tg mice were immunized with a peptide containing the human disease-associated B cell epitope (LYQEARKIVGAMVQITTYR (SEQ ID NO: 35), the human and mouse sequences are identical). Compared with OVA aa323-339 immunized mice, MPO aa442-460 immunized mice developed albuminuria, hematuria, had increased BUN values and mild glomerular proliferation (FIG. 8). Pooled serum IgG purified from these mice showed a p-ANCA pattern when applied to normal Mpo^{+/+}, but not Mpo^{-/-} neutrophils (FIG. 8A). This IgG pool was passively transferred into naïve DR2 Tg mice treated with LPS. When studied one and six days after IgG transfer, recipients of anti-MPO antibodies from MPO aa442-460 immunized donors developed albuminuria, some early hematuria and a raised BUN (FIG. 9A-9C). IgG induced glomerular injury (FIGS. 9D and 9E) and neutrophil recruitment after transfer to (FIG. 9F). Each recipient mouse developed glomerular histological abnormalities marked by hypercellularity with glomerular neutrophil accumulation, producing a proliferative, albeit not a necrotizing lesion. Immunizing MHC II humanized mice with aa442-460 (conserved between human and mice) induced a proliferative glomerular disease and MPO-reactive antibodies after 28 days. A causal role for MPO-reactive antibodies was supported by disease of greater severity upon transfer of Ig from the immunized animals into naïve recipients.

As HLA-MHC II help shape the B and T cell repertoires, and consequently the response to foreign and self-antigens, we wanted to define the epitope specificity of the anti-MPO autoantibodies that could be induced experimentally using DR2 Tg mice. In this way, we could assess development of the anti-MPO antibody response after immunization of a single epitope in the context of a human MHC II. IgG from sera of immunized mice was profiled for anti-MPO epitope specificity using epitope excision MS. DR2 Tg mice developed antibodies targeting the human B cell epitope aa447-459 which is within a conserved region of both murine and human MPO. Unpredictably, immunized DR2 Tg mice initiated a polyclonal response producing autoantibodies against the entire MPO molecule. The epitope profile was astonishingly similar to that of the MPO-ANCA human profile (Table 6), indicative of the critical nature of this epitope.

TABLE 6
Comparison of human and mice MPO epitopes upon immunization
and passive transfer of MPO epitope aa442-460

| | | | Found in samples: | |
|---|---|---|---|---|
| | | | Healthy control, | |
| Epitope A | | | Active disease, | |
| Sequence | Epitopes found | Epitopes found in | or Remission, | |
| Number | in humans | immunized mice | (HC, A, R) | Epitope Structure |
| 490-499 | IANVFTNAFR | IANVFTNAFR | A | Conformational |
| | (SEQ ID NO: 1) | (SEQ ID NO: 1) | | |
| 537-548 | VVLEGGIDPILR | VVLEGGIDPILR | A | Conformational |
| | (SEQ ID NO: 2) | (SEQ ID NO: 2) | | |
| 328-351 | NQINALTSFVDASMV | | A | Conformational |
| | (SEQ ID NO: 3) | | | |
| 220-228 | NGFPVALAR | | A | Conformational |
| | (SEQ ID NO: 4) | | | |
| 198-219 | WLPAEYEDGFSLPYG | | A | Conformational |
| | (SEQ ID NO: 5) | | | |
| 447-459 | RKIVGAMVQIITY | RKIVGAMVQIITY | A | Linear |
| | (SEQ ID NO: 6) | (SEQ ID NO: 6) | | |
| 369-374 | FQDNGR | | A | Conformational |
| | (SEQ ID NO: 7) | | | |
| 184-193 | RSPTLGASNR | RSPTLGASNR | A | Conformational |
| | (SEQ ID NO: 8) | (SEQ ID NO: 8) | | |
| 605-622 | FCGLPQPETVGQLGT | | A | Conformational |
| | (SEQ ID NO: 9) | | | |
| 442-447 | LYQEAR | | A | Conformational |
| | (SEQ ID NO: 10) | | | |
| 715-725 | NNIFMSNTYPR | NNIFMSNTYPR | A | Conformational |
| | (SEQ ID NO: 11) | (SEQ ID NO: 11) | | |
| 657-664 | VGPLLACI | | A | Conformational |
| | (SEQ ID NO: 12) | | | |
| 560-571 | QNQIAVDEIR | | A, R | Conformational |
| | (SEQ ID NO: 13) | | | |
| 692-701 | QALAQISLPR | | A, R | Conformational |
| | (SEQ ID NO: 14) | | | |
| 474-480 | KYLPTYR | | A, R | Conformational |
| | (SEQ ID NO: 15) | | | |
| 437-441 | WDGER | | A, R | Conformational |
| | (SEQ ID NO: 16) | | | |
| 396-405 | IPCFLAGDTR | IPCFLAGDMR | R | Conformational |
| | (SEQ ID NO: 17) | (SEQ ID NO: 36) | | |
| 516-524 | YQPMEPNPR | | A, R, HC | Linear |
| | (SEQ ID NO: 18) | | | |
| 579-590 | IGLDLPALNMQR | IGLDLPALNMQR | A, R, HC | Linear |
| | (SEQ ID NO: 19) | (SEQ ID NO: 19) | | |
| 530-536 | VFFASWR | VFFASWR | A, R, HC | Linear |
| | (SEQ ID NO: 20) | (SEQ ID NO: 20) | | |
| 237-248 | FPTDQLTPDQER | | A, R, HC | Linear |
| | (SEQ ID NO: 21) | | | |
| 460-473 | RDYLPLVLGPTAMR | | A, R, HC | Conformational |
| | (SEQ ID NO: 22) | | | |
| 593-603 | DHGLPGYNAWR | DHGLPGYNAWR | A, R, HC | Conformational |
| | (SEQ ID NO: 23) | (SEQ ID NO: 23) | | |
| 572-578 | LFEQVMR | LFEQVMR | A, R, HC | Conformational |
| | (SEQ ID NO: 24) | (SEQ ID NO: 24) | | |
| 678-691 | FWWENEGVFSMQQR | | A, R, HC | Conformational |
| | (SEQ ID NO: 26) | | | |

Fundamental questions problematic to the understanding of ANCA vasculitis were resolved, at least in part, by the studies presented here. These studies explain why asymptomatic or ‘natural’ MPO autoantibodies can exist in individuals free of disease: epitope specificity defines pathogenicity. They explain why there is a lack of correlation between MPO-ANCA titers and active disease: clinical tests do not discriminate between natural anti-MPO autoantibodies and potentially pathogenic ANCA. They explain why some patients with vasculitis are seronegative for ANCA in clinical tests: detection of a monoclonal MPO-ANCA reactive with a small, restricted epitope is obstructed by a protein present in sera.

For years, ANCA-negative small vessel vasculitis has posed a clinical and pathogenic dilemma. Our findings suggest that they develop a restricted autoantibody response against a linear epitope on MPO (aa447-459), an epitope masked by a ceruloplasmin fragment which is crucial concerning this group of patients. Clinically, patients with ANCA-negative SVV have the same signs and symptoms of disease as those patients who are ANCA-positive (Chen et al., Nature Rev. Nephrol. 5:313 (2009); Chen et al., J. Am. Soc. Nephrol. 18:599 (2007)). Frequently, these patients are diagnosed late in the course of their illness. Many of these patients have reoccurring ear, nose and throat disease. We were surprised that many, but not all, of the UNC and Groningen cohorts who are ANCA-negative had an antibody to MPO and not to proteinase 3. From a pathogenic perspective, ANCA-negative patients presented a real quandary to those who propose that ANCA are fundamental to the pathogenesis of pauci-immune small vessel vasculitis. The findings in this study, that a portion of ANCA-negative patients have MPO-ANCA, diminish that concern. The linear epitope identified is the same one that was found to be associated with active disease in the MPO-ANCA positive patient group, which declined upon clinical remission. These findings underscore the pathogenic capacity of antibodies against this epitope, further supported by their ability to activate neutrophils in vitro and cause nephritogenic disease in a transgenic murine model. These observations are supported by in vivo murine data that an autoantibody to murine MPO epitope aa409-428 or overlapping human MPO epitope aa442-460 (Ooi et al., Proc. Natl. Acad. Sci. USA 109:E2615 (2012)) may be the instigating event that triggers a polyclonal autoantibody profile, similar to an MPO-positive patient's polyclonal repertoire during active disease (Table 6).

Anti-MPO⁴⁴⁷⁻⁴⁵⁹ autoantibodies are exclusive to active disease. An enzymatically-produced fragment of a natural host protein, abundant in serum, shields MPO epitope aa447-459 from autoantibody binding. An observation that remains a puzzle is why only enzymatically processed ceruloplasmin fragments have the epitope “blocking” capability, while full-length protein does not. We used plasmin to generate a fragment of a size that correlated with that purified from serum, mainly because plasmin has been reported to cleave ceruloplasmin (Kingston et al., Proc. Natl. Acad. Sci. USA 74:5377 (1977)). We tested other serum proteases in which urokinase produced a functional fragment but thrombin did not, underscoring the specificity of the “blocking” mechanism. Full-length ceruloplasmin is the natural inhibitor of MPO activity and it was initially suggested that patients diagnosed with MPO-ANCA might be deficient in ceruloplasmin. Subsequent reports determined the converse to be true, that ANCA patients did not have a deficiency in serum ceruloplasmin levels and instead had a marked increase during active disease (Ara et al., Nephrol. Dial. Transplant. 14:515 (1999); Baskin et al., Pediatr. Nephrol. 17:917 (2002)). In addition, ANCA were reported to have the ability to disassociate the MPO/ceruloplasmin complex (Griffin et al., Kidney Int. 55:917 (1999)). However, anti-MPO⁴⁴⁷⁻⁴⁵⁹ autoantibodies did not dislodge the complex of MPO peptide aa447-459 and the ceruloplasmin fragment in our assays, accentuating the high affinity of the complex.

Researchers who have studied anti-MPO specific epitopes agreed that most epitopes were likely conformationally-dependent, although a few linear epitopes had been identified. Our data verify and extend the epitope mapping literature. The light chain of MPO was reported to contain immunodominant linear epitopes recognized by MPO-ANCA sera samples (Pedrollo et al., Adv. Exp. Med. Biol. 336:87 (1993)). We too found a linear epitope in the light chain (aa237-248) of MPO and along with three conformational epitopes. Conformationally-dependent epitopes were reported to lie within two regions of the heavy chain (aa279-341 and aa474-512) (Fujii et al., Clin. Nephrol. 53:242 (2000); Tomizawa et al., J. Clin. Immunol. 18:142 (1998); Suzuki et al., Microbiol. Immunol. 51:1215 (2007)), which overlap three disease-associated conformational epitopes we identify here. Using MPO human/mouse chimeras, aa517-667 and aa668-774 were identified as important epitopes (Erdbrugger et al., Kidney Int. 69:1799 (2006)). We too identified 12 epitopes that fall within these two segments. Another group used overlapping peptide ELISAs and identified seven linear immunodominant epitopes (Bruner et al., Clin. Exp. Immunol. 164:330 (2011)). One of these, aa511-522 (RLDNRYQPMEPN (SEQ ID NO: 30)), overlapped with one we identified as a disease-associated epitope, aa516-524 (YQPMEPNPR (SEQ ID NO: 18)).

Asymptomatic or ‘natural’ autoantibodies are known to occur prior to the onset of autoantibody-induced ANCA and anti-GBM disease (Cui et al., Kidney Int. 78:590 (2010); Olson et al., J. Am. Soc. Nephrol. 22:1946 (2011); Xu et al., Rheumatology (Oxford) 50:1236 (2011)). Visualizing epitopes on the three dimensional crystal structure of MPO (pdb 3f9) revealed an interesting adjacency of ‘natural’ epitopes to epitopes exclusive to active disease (FIG. 3C). This suggests the possibility that an asymptomatic autoantibody response to MPO may precede the onset of autoimmune disease (Cui et al., Kidney Int. 78:590 (2010); Olson et al., J. Am. Soc. Nephrol. 22:1946 (2011); Xu et al., Rheumatology (Oxford) 50:1236 (2011)) and that epitope spreading to epitopes exclusive to active disease may be one step of a cascade that initiates disease onset. It is also conceivable that asymptomatic autoantibodies against MPO could structurally alter the conformation of adjacent epitopes and expose cryptic epitopes that would induce potentially pathogenic autoantibodies.

In summary, these observations demonstrate substantial MPO-ANCA epitope diversity. Three functionally distinct classes of autoantibody/epitope pairs are recognized: epitopes exclusive to active disease, persistent during remission, and asymptomatic or ‘natural’. While titers of total MPO-ANCA do not correlate well with disease activity, specific titers of MPO-ANCA directed against epitopes exclusive to active disease correlate extremely well with disease activity. For the first time, the perplexing ANCA-negative subset of AAV patients is explained by identifying occult MPO-ANCA that have very restricted specificity for an anti-MPO autoantibody that is blocked in serum by a ceruloplasmin fragment.

The foregoing is illustrative of the present invention, and is not to be construed as limiting thereof. The invention is defined by the following claims, with equivalents of the claims to be included therein.

All publications, patent applications, patents and other references cited herein are incorporated by reference in their entireties for the teachings relevant to the sentence and/or paragraph in which the reference is presented.

1. An isolated polypeptide comprising a peptide as shown in Table 1 or an equivalent thereof that is specifically bound by a myeloperoxidase anti-neutrophil cytoplasmic autoantibody (MPO-ANCA) that specifically binds to the peptide of Table 1.

2. The polypeptide of claim 1, wherein the polypeptide comprises a MPO peptide consisting of a peptide as shown in Table 1 or an equivalent thereof that is specifically bound by MPO-ANCA that specifically binds to the peptide of Table 1.

3. The polypeptide of claim 1, bound to a solid support.

4. The polypeptide of claim 3, wherein the solid support is a bead, particle, membrane, plate, slide, chip, or well.

5. A kit comprising the polypeptide of claim 1.

6. A method of detecting the presence of MPO-ANCA in a biological sample from a mammalian subject, the method comprising:
contacting the sample with the polypeptide of claim 1; and
detecting binding of MPO-ANCA in the sample to the polypeptide, wherein binding of MPO-ANCA to the polypeptide indicates the presence of MPO-ANCA in the sample.

7. A method of diagnosing a mammalian subject with an autoimmune disorder characterized by the presence of MPO-ANCA and/or determining if the subject is at risk for the autoimmune disorder, the method comprising:
contacting a biological sample from the subject with the polypeptide of claim 1; and
detecting binding of MPO-ANCA in the sample to the polypeptide, wherein binding of MPO-ANCA to the polypeptide diagnoses the subject with the autoimmune disorder and/or indicates that the subject is at risk for the autoimmune disorder.

8. A method of monitoring disease progression in a mammalian subject with an autoimmune disorder characterized by the presence of MPO-ANCA, the method comprising:
contacting a biological sample from the subject with the polypeptide of claim 1; and
detecting binding of MPO-ANCA in the sample to the polypeptide, wherein binding of MPO-ANCA to the polypeptide correlates with disease progression in the subject.

9. A method of determining whether a mammalian subject is in remission from an autoimmune disorder characterized by the presence of MPO-ANCA, the method comprising:
contacting a biological sample from the subject with the polypeptide of claim 1; and
detecting binding of MPO-ANCA in the sample to the polypeptide, wherein binding of MPO-ANCA to the polypeptide provides information regarding whether the subject is in remission.

10. The method of claim 6, wherein the subject is a human subject.

11. The method of claim 10, wherein the subject has or is suspected of having MPO-ANCA glomerulonephritis.

12. The method of claim 10, wherein the subject has or is suspected of having ANCA-negative glomerulonephritis.

13. The method of claim 6, wherein the biological sample is a blood sample.

14. The method of claim 13, wherein the blood sample has been processed to enrich for immunoglobulins (Ig).

15. The method of claim 14, wherein the blood sample has been processed to enrich for IgG, IgA and/or IgM.

16. The method of claim 13, wherein the blood sample has been processed to reduce ceruloplasmin or the 45 kilodalton cleavage product of ceruloplasmin.

17. The method of claim 6, wherein the detecting step comprises an immunoassay.

18. The method of claim 17, wherein the immunoassay comprises immunopurification, an Enzyme Immunoassay (EIA), an Enzyme Linked-Immunosorbent assay (ELISA), Western blot and/or immunofluorescence.

19. The method of claim 6, wherein the detection step comprises mass spectrometry.


SEQUENCE LISTING (37 sequences)

SEQ ID NO: 1 - protein, 10 aa, Homo sapiens
IANVFTNAFR

SEQ ID NO: 2 - protein, 12 aa, Homo sapiens
VVLEGGIDPILR

SEQ ID NO: 3 - protein, 15 aa, Homo sapiens
NQINALTSFVDASMV

SEQ ID NO: 4 - protein, 9 aa, Homo sapiens
NGFPVALAR

SEQ ID NO: 5 - protein, 15 aa, Homo sapiens
WLPAEYEDGFSLPYG

SEQ ID NO: 6 - protein, 13 aa, Homo sapiens
RKIVGAMVQIITY

SEQ ID NO: 7 - protein, 6 aa, Homo sapiens
FQDNGR

SEQ ID NO: 8 - protein, 10 aa, Homo sapiens
RSPTLGASNR

SEQ ID NO: 9 - protein, 15 aa, Homo sapiens
FCGLPQPETVGQLGT

SEQ ID NO: 10 - protein, 6 aa, Homo sapiens
LYQEAR

SEQ ID NO: 11 - protein, 11 aa, Homo sapiens
NNIFMSNTYPR

SEQ ID NO: 12 - protein, 8 aa, Homo sapiens
VGPLLACI

SEQ ID NO: 13 - protein, 10 aa, Homo sapiens
QNQIAVDEIR

SEQ ID NO: 14 - protein, 10 aa, Homo sapiens
QALAQISLPR

SEQ ID NO: 15 - protein, 7 aa, Homo sapiens
KYLPTYR

SEQ ID NO: 16 - protein, 5 aa, Homo sapiens
WDGER

SEQ ID NO: 17 - protein, 10 aa, Homo sapiens
IPCFLAGDTR

SEQ ID NO: 18 - protein, 9 aa, Homo sapiens
YQPMEPNPR

SEQ ID NO: 19 - protein, 12 aa, Homo sapiens
IGLDLPALNMQR

SEQ ID NO: 20 - protein, 7 aa, Homo sapiens
VFFASWR

SEQ ID NO: 21 - protein, 12 aa, Homo sapiens
FPTDQLTPDQER

SEQ ID NO: 22 - protein, 14 aa, Homo sapiens
RDYLPLVLGPTAMR

SEQ ID NO: 23 - protein, 11 aa, Homo sapiens
DHGLPGYNAWR

SEQ ID NO: 24 - protein, 9 aa, Homo sapiens
YQPMEPNPR

SEQ ID NO: 25 - protein, 7 aa, Homo sapiens
LFEQVMR

SEQ ID NO: 26 - protein, 14 aa, Homo sapiens
FWWENEGVFSMQQR

SEQ ID NO: 27 - protein, 6 aa, Homo sapiens
  misc_feature (3)..(3): Xaa can be any naturally occurring amino acid
RKXVGA

SEQ ID NO: 28 - protein, 6 aa, Homo sapiens
RKIVGA

SEQ ID NO: 29 - protein, 5 aa, Homo sapiens
VVLEG

SEQ ID NO: 30 - protein, 12 aa, Homo sapiens
RLDNRYQPMEPN

SEQ ID NO: 31 - protein, 7 aa, Homo sapiens
YQPMEPN

SEQ ID NO: 32 - protein, 14 aa, Homo sapiens
RKIVGAMVQIITYR

SEQ ID NO: 33 - protein, 13 aa, Homo sapiens
DYLPLVLGPTAMR

SEQ ID NO: 34 - protein, 20 aa, Homo sapiens
PRWNGEKLYQEARKIVGAMV

SEQ ID NO: 35 - protein, 19 aa, Homo sapiens
LYQEARKIVGAMVQITTYR

SEQ ID NO: 36 - protein, 10 aa, Mus musculus
IPCFLAGDMR

SEQ ID NO: 37 - protein, 745 aa, Homo sapiens
MGVPFFSSLRCMVDLGPCWAGGLTAEMKLLLALAGVLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS